(19) United States
(12) Patent Application Publication
(10) Pub. No.: US 2022/0254466 A1
(43) Pub. Date: Aug. 11, 2022

(54) SYSTEMS AND METHODS FOR AUTOMATED PRIOR AUTHORIZATION

(71) Applicant: Flatiron Health, Inc., New York, NY (US)

(72) Inventors: Andrew HWANG, Brooklyn, NY (US); Rebecca MANIAGO, Goshen, KY (US); Harvey James HARMRICK, Jr., Atlanta, GA (US); Michael Tyler HAYDELL, New York, NY (US); Shawn HUDA, New York, NY (US)

(73) Assignee: Flatiron Health, Inc., New York, NY (US)

(21) Appl. No.: 17/172,914

(22) Filed: Feb. 10, 2021

(51) Int. Cl. G16H 20/00; G16H 10/60; G06Q 40/08; H04L 29/06

(52) U.S. Cl. CPC G16H 20/00; H04L 67/40; G06Q 40/08; G16H 10/60

(57) ABSTRACT

Systems and methods for computer-based automated pre-approvals are disclosed. A system may comprise at least one processing device configured to access information from at least one database via the network interface, generate a first graphical user interface (GUI) for receiving selection of a treatment regimen, generate a first electronic message for seeking pre-approval of the selected treatment regimen based on the selected treatment regimen and the accessed information, and format the first electronic message for transmission through an application programming interface (API). The system may transmit, via the network interface and the API, the first electronic message to an insurance provider system, receive, from the insurance provider system via the network interface and the API, a second electronic message. The system may extract, from the second electronic message, approval information, and generate a second GUI for providing the extracted approval information.

### CROSS-REFERENCES TO RELATED APPLICATIONS

This application is related to U.S. application Ser. No. 16/703,113, filed Dec. 3, 2019, which claims the benefit of U.S. Provisional Application No. 62/774,621, filed Dec. 3, 2018, which are incorporated by reference in their entireties.

### BACKGROUND

### Technical Field

The present disclosure relates to systems and methods for generating computer-based healthcare treatment approval. More particularly, the disclosed embodiments relate to rules-based approval techniques using dynamic graphical user interfaces and distributed, networked computer systems for seeking prior authorization for treatment, and guideline concordance.

### Background Information

Patients undergoing treatment for illnesses generally rely on insurance companies to pay the treatment costs. Prior to paying, however, the insurance company must approve the treatment and associated costs. In some situations, the insurance company (also referred to herein as the “payer”) accepts or declines payment after treatment, potentially shifting the financial burden to the patient. Due to the risk of declined insurance coverage, caregivers usually seek pre-approval for treatment and medication costs.

The traditional pre-approval process involves numerous rounds of communications between the caregiver and the insurance provider. The caregiver first sends information about the patient and the condition, and then the insurance provider may request additional supporting information, requiring the caregiver to collect the information and engage in additional rounds of communication. This iterative process is time consuming, prone to error, inconsistencies, and does not leverage the capabilities of modern computing and communications systems.

To increase the consistency in patient treatment and insurance coverage, guidelines have been developed by authoritative professional societies and associations. One example of a well-known provider of guidelines for oncology is the National Comprehensive Cancer Network (NCCN), but other institutions publish their own guidelines as well. Guidelines can assist caregivers with making informative decisions about how to treat complex and specific diseases and mutations. The guidelines include decision-making tools that provide evidence-based standards to help providers determine how to best treat a patient based on the patient's unique clinical factors, such as disease, stage, disease status and more.

Health systems typically need to report clinical information to insurance companies and other payers to justify treatment decisions. The NCCN guidelines are a widely used source to determine what clinical data points are required to justify a given treatment decision. Physicians do not currently have a tool at the point of care to efficiently capture clinical information associated with guideline concordance and to request payer pre-authorization. For example, to obtain guideline-concordant treatment recommendations, physicians must navigate out of a typical workflow in a point of care platform (e.g., an electronic health records software solution) to review numerous pdf documents or to request payer pre-authorization via email or phone. This can create unnecessary time delays for obtaining payer approval for patient's treatment regimen and disrupt the physician's workflow.

Accordingly, there is a need for improvements in to point of care solutions and cost approval/pre-approval techniques.

### SUMMARY

Disclosed embodiments provide new computerized techniques for healthcare insurance approvals and pre-approvals, that leverage the data processing and communication capabilities of modern computing systems. Disclosed embodiments also fully integrate a treatment regimen ordering and approval workflow within a caregiver's workflow and interface. enable better guideline concordance reporting with minimal disruption to physician workflow and a better user experience around treatment selection through decision support. Such a point of care solution would facilitate data collection, thereby enabling retrospective analysis, and would support physician decision-making around treatment recommendations.

In an embodiment, a system for computer-based automated pre-approvals may comprise: a memory device storing instructions; a network interface; and at least one processing device in communication with the memory device and the network interface. The at least one processing device may be configured to execute the stored instructions to access, via the network interface, information from at least one database; generate a first graphical user interface (GUI) for receiving selection of a treatment regimen; generate, based on the selected treatment regimen and the accessed information, a first electronic message for seeking pre-approval of the selected treatment regimen; format the first electronic message for transmission through an application programming interface (API); transmit, via the network interface and the API, the first electronic message to an insurance provider system; receive, from the insurance provider system via the network interface and the API, a second electronic message; extract, from the second electronic message, approval information; and generate a second GUI for providing the extracted approval information.

In an embodiment a computer-implemented method for automated pre-approval may include accessing, via a network interface, information from at least one database; generating a first graphical user interface (GUI) for receiving selection of a treatment regimen; generating, based on the selected treatment regimen and the accessed information, a first electronic message for seeking pre-approval of the selected treatment regimen; formatting the first electronic message for transmission through an application programming interface (API); transmitting, via the API, the first electronic message to an insurance provider system; receiving, from the insurance provider system, a second electronic message; extracting, from the second electronic message, approval information; and generating a second GUI for providing the extracted approval information.

Consistent with other disclosed embodiments, non-transitory computer readable storage media may store program instructions, which are executed by at least one processing device and perform any of the methods described herein.

### BRIEF DESCRIPTION OF THE DRAWINGS

The accompanying drawings, which are incorporated in and constitute part of this specification, and together with the description, illustrate and serve to explain the principles of various exemplary embodiments. In the drawings:

FIG. 1 is a block diagram illustrating an exemplary system environment for implementing embodiments consistent with the present disclosure.

FIG. 2 is a block diagram illustrating an exemplary process for implementing embodiments consistent with the present disclosure.

FIG. 3 is an illustration of an exemplary graphical user interface (GUI) consistent with the present disclosure.

FIG. 4A-4D are illustrations of exemplary GUIs consistent with the present disclosure.

FIG. 5 is an illustration of an exemplary GUI consistent with the present disclosure.

FIG. 6 is a flowchart illustrating an exemplary process for providing guideline concordance consistent with the present disclosure.

FIG. 7 is an illustration of an exemplary regimen selection GUI consistent with the present disclosure.

FIGS. 8 and 9 are illustrations of exemplary approval GUIs consistent with the present disclosure.

FIG. 10 is an illustration of an exemplary flowsheet generation GUI consistent with the present disclosure.

FIG. 11 is an illustration of an exemplary treatment plan GUI consistent with the present disclosure.

FIGS. 12 and 13 are illustrations of exemplary prior authorization GUIs consistent with the present disclosure.

FIG. 14 is an illustration of an exemplary biosimilar GUI consistent with the present disclosure.

FIG. 15 is an illustration of another exemplary flowsheet generation GUI consistent with the present disclosure.

FIG. 16 is an illustration of another exemplary treatment plan GUI consistent with the present disclosure.

FIG. 17 is an illustration of another exemplary prior authorization GUI consistent with the present disclosure.

FIG. 18 is an illustration of another exemplary drug order GUI consistent with the present disclosure.

FIG. 19 is a flowchart illustrating an exemplary process for providing automated prior authorization consistent with the present disclosure.

### DETAILED DESCRIPTION

The following detailed description refers to the accompanying drawings. Wherever possible, the same reference numbers are used in the drawings and the following description to refer to the same or similar parts. While several illustrative embodiments are described herein, modifications, adaptations and other implementations are possible. For example, substitutions, additions or modifications may be made to the components illustrated in the drawings, and the illustrative methods described herein may be modified by substituting, reordering, removing, or adding steps to the disclosed methods. Accordingly, the following detailed description is not limited to the disclosed embodiments and examples. Instead, the proper scope is defined by the appended claims.

Embodiments herein include computer-implemented methods, tangible non-transitory computer-readable mediums, and systems. The computer-implemented methods may be executed, for example, by at least one processor (e.g., a processing device) that receives instructions from a non-transitory computer-readable storage medium. Similarly, systems consistent with the present disclosure may include at least one processor (e.g., a processing device) and memory, and the memory may be a non-transitory computer-readable storage medium. As used herein, a non-transitory computer-readable storage medium refers to any type of physical memory on which information or data readable by at least one processor may be stored. Examples include random access memory (RAM), read-only memory (ROM), volatile memory, nonvolatile memory, hard drives, CD ROMs, DVDs, flash drives, disks, and any other known physical storage medium. Singular terms, such as “memory” and “computer-readable storage medium,” may additionally refer to multiple structures, such a plurality of memories and/or computer-readable storage mediums. As referred to herein, a “memory” may comprise any type of computer-readable storage medium unless otherwise specified. A computer-readable storage medium may store instructions for execution by at least one processor, including instructions for causing the processor to perform steps or stages consistent with an embodiment herein. Additionally, one or more computer-readable storage mediums may be utilized in implementing a computer-implemented method. The term “computer-readable storage medium” should be understood to include tangible items and exclude carrier waves and transient signals.

Embodiments of the present disclosure provide systems and methods for providing guideline concordance. A user of the disclosed systems and methods may encompass any individual who may wish to access a patient's clinical experience and/or analyze patient data. Thus, throughout this disclosure, references to a “user” of the disclosed systems and methods may encompass any individual, such as a physician, a clinician, a quality assurance department at a health care institution, and/or the patient. While reference is made to tumors or cancer therapies throughout this disclosure, these are provided as an example only, and it is understood that the disclosed systems and methods may apply to various other diseases and/or treatments.

Disclosed embodiments describe a point of care solution that enables better guideline concordance reporting with minimal disruption to physician workflow and better user experience around treatment selection through decision support. For example, the user interface may be the means through which a physician can report guideline concordance without having to use a pathways tool, thereby improving efficiency and providing a seamless process. Pathway tools are often inefficient and require the user to navigate through one or more pdf documents outside the workflow. Additionally, in some embodiments, the user interface may improve the overall treatment selection by providing clinical decision support. Disclosed embodiments may provide an application that seamlessly captures patient data points, which may, in turn, accelerate reimbursement approval, enhance negotiating strength, and reduce operational costs for cancer centers. Additionally, disclosed embodiments may provide an application that seamlessly packages the patient data points for providing to a third party, such as an insurance provider, to automate and accelerate the reimbursement approval process.

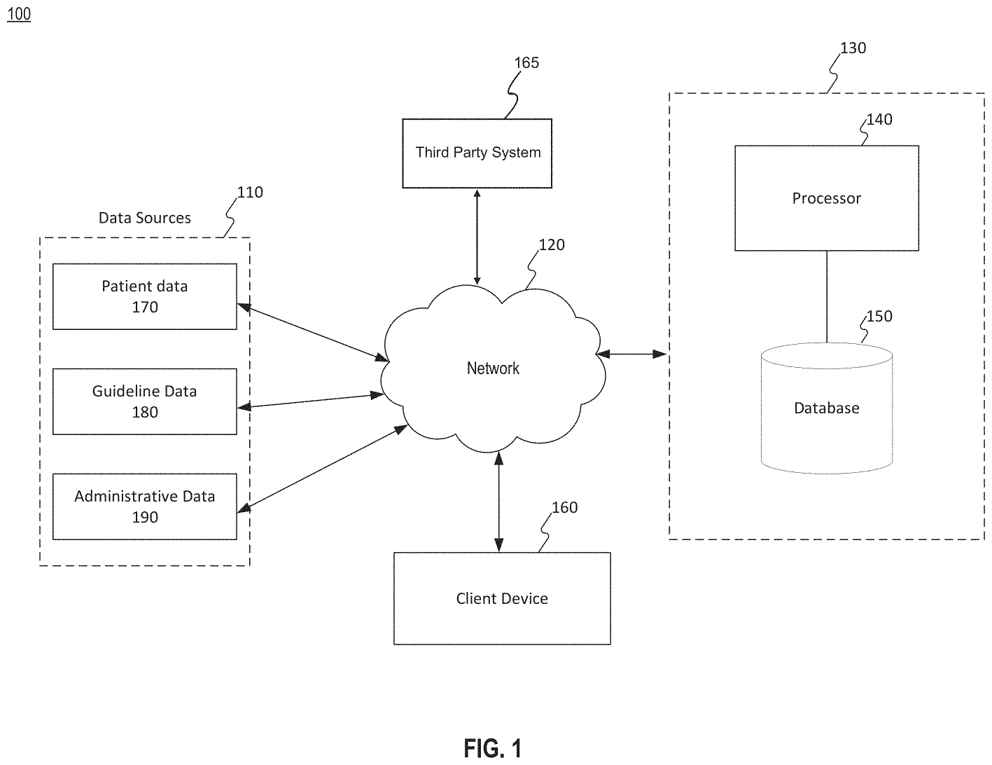

FIG. 1 illustrates an exemplary system environment 100 for implementing embodiments consistent with the present disclosure, described in detail below. As shown in FIG. 1, system environment 100 includes several components. It will be appreciated from this disclosure that the number and arrangement of these components is exemplary and provided for purposes of illustration. Other arrangements and numbers of components may be used without departing from the teachings and embodiments of the present disclosure.

As shown in FIG. 1, exemplary system environment 100 includes a system 130. System 130 may include one or more server systems, databases, and/or computing systems configured to receive information from entities over a network, process the information, store the information, and display/transmit the information to other entities over the network. Thus, in some embodiments, the network may facilitate cloud sharing, storage, and/or computing. In one embodiment, system 130 may include a processor 140 and one or more databases 150, which are illustrated in a region bounded by a dashed line representing system 130 in FIG. 1. Processor 140 may comprise at least one processing device, such as one or more generic processors, e.g., a central processing unit (CPU), a graphics processing unit (GPU), or the like and/or one or more specialized processors, e.g., an application-specific integrated circuit (ASIC), a field-programmable gate array (FPGA), or the like.

In one embodiment, system 130 may include a network interface for transmitting and/or receiving data to/from various other components, such as one or more data sources 110 and client device 160. More specifically, system 130 may be configured to receive and store the data transmitted over a network 120 (e.g., the Internet, an Intranet, WAN, LAN, cellular network, Bluetooth, etc.) from various data sources, including data sources 110, process the received data, and transmit data and results based on the processing to client device 160.

The various components of system environment 100 may include an assembly of hardware, software, and/or firmware, including a memory, a central processing unit (CPU), and/or a user interface. Memory may include any type of RAM or ROM embodied in a physical storage medium, such as magnetic storage including floppy disk, hard disk, or magnetic tape; semiconductor storage such as solid-state disk (SSD) or flash memory; optical disc storage; or magneto-optical disc storage. A CPU may include one or more processors for processing data according to a set of programmable instructions or software stored in the memory. The functions of each processor may be provided by a single dedicated processor or by a plurality of processors. Moreover, processors may include, without limitation, digital signal processor (DSP) hardware, or any other hardware capable of executing software. An optional user interface may include any type or combination of input/output devices, such as a display monitor, keyboard, and/or mouse.

Data transmitted and/or exchanged with or within system environment 100 may occur over a data interface. As used herein, a data interface may include any boundary across which two or more components of system environment 100 exchange data. For example, environment 100 may exchange data between software, hardware, databases, devices, humans, or any combination of the foregoing. Furthermore, it will be appreciated that any suitable configuration of software, processors, data storage devices, and networks may be selected to implement the components of system environment 100 and features of related embodiments.

As described further below, system 130 may be configured to receive patient data 170, guideline data 180, and/or administrative data 190, e.g., data indicating preferred treatments or available trials, from data sources 110 or other sources in network 120. Data sources 110 may include a variety of sources of medical, guideline, and administrative data. For example, data sources 110 may include medical care providers of the patient, such as physicians, nurses, specialists, consultants, hospitals, clinics, and the like. Data sources 110 may also include laboratories such as radiology or other imaging labs, hematology labs, pathology labs, etc. Data sources 110 may also include any other sources of patient, insurance, or guideline data.

In patient data 170, each patient may be represented by one or more records generated by one or more health care professionals or by the patient. For example, a doctor associated with the patient, a nurse associated with the patient, a physical therapist associated with the patient, or the like, may each generate a medical record for the patient. In some embodiments, one or more records may be collated and/or stored in the same database. In other embodiments, one or more records may be distributed across a plurality of databases. In some embodiments, the records may be stored and/or provided a plurality of electronic data representations. For example, the patient records may be represented as one or more electronic files, such as text files, portable document format (PDF) files, extensible markup language (XML) files, or the like. If the documents are stored as PDF files, images, or other files without text, the electronic data representations may also include text associated with the documents derived from an optical character recognition process. In some embodiments, patient data may be stored in structured database records, unstructured database records, or a combination thereof. Structured data may be organized, addressed, indexed, or formatted so that the data is searchable in relational database records. Unstructured data may include data stored in a native format, and without an organizational model or defined set of formats. Patient data 170 may be stored, for example, as electronic health records (EHRs) in an electronic health record database. In some embodiments, a patient EHR may include, for example, identification information (e.g., name, address, date of birth, etc.), medical history information (e.g., treatment dates, surgical history, prescribed medicines, family medical history, etc.), provider information (e.g., primary insurance provider, copay amount, secondary insurance provider), and/or contact information (e.g., emergency contact information, primary care provider contact information, etc.).

In some embodiments, guideline data 180 may be received from a guideline publisher, e.g., NCCN. Guideline data 180 may include a guideline compendium storing non-structured data. For example, a compendium may store unstructured decision tree data in a tabular format. In some embodiments, guideline data 180 may include one or more decision trees defining guidelines and/or pathways associating clinical attributes and/or indications with one or more treatment regimens. In some embodiments, one or more guideline documents may be collated and/or stored in the same database. In other embodiments, one or more guideline documents may be distributed across a plurality of databases. In some embodiments, the guidelines may be stored and/or provided a plurality of electronic data representations. For example, the guidelines may be represented as one or more electronic files, such as text files, portable document format (PDF) files, extensible markup language (XML) files, or the like. If the documents are stored as PDF files, images, or other files without text, the electronic data representations may also include text associated with the documents derived from an optical character recognition process.

Administrative data 190 may be data received from an insurance provider or medical personnel, e.g., hospital administrator. The administrator data may indicate one or more preferred pathways or may indicate treatments covered by a patient's insurance. In some embodiments, insurance data for a particular patient may be provided as patient data 170. Administrative data 190 may be received from an administrative system, e.g., a hospital system or hospital database and may be represented as one or more electronic files, such as text files, portable document format (PDF) files, extensible markup language (XML) files, or the like. If the documents are stored as PDF files, images, or other files without text, the electronic data representations may also include text associated with the documents derived from an optical character recognition process.

System 130 may further communicate with one or more client devices 160 over network 120. For example, system 130 may provide a list of regimens in response to a search performed by a physician based on analysis of data from data sources 110 to client device 160. Client device 160 may include any entity or device capable of receiving or transmitting data over network 120. For example, client device 160 may include a computing device, such as a server or a desktop or laptop computer. Client device 160 may also include other devices, such as a mobile device, a tablet, a wearable device (i.e., smart watches, implantable devices, fitness trackers, etc.), a virtual machine, an IoT device, or other various technologies. In some embodiments, client device 160 may transmit queries for information about a patient and/or about a treatment over network 120 to system 130, such as a query for a particular treatment, patient medical records, or various other information about a patient.

Guideline data 180 may be received from data sources 110 and processed by system 130, as described above. The guidelines received from data sources 110 (or elsewhere) may include one or more decision trees. The decision trees may guide a provider through the process of selecting a treatment based on a patient's clinical attributes. Traditionally, a provider navigates through a number of decision trees stored as pdf documents to identify one or more guideline concordant treatments for the patient based on the patient's clinical attributes. This method may disrupt the practitioner workflow, resulting in inefficiencies and inconsistent tracking of guideline concordance. For example, using guideline decision trees may require a practitioner to navigate numerous pdfs and make a number of decisions before arriving at a treatment recommendation. Further, this traditional approach lacks flexibility since the practitioner is required to follow an ordered sequence of decisions.

In some embodiments, system environment 100 may include one or more of third party system 165, such as one or more computer systems associated with an insurance provider. Third party system 165 may be connected to network 120 and to other devices in system environment 100 via network 120. In some embodiments, Third party system 165 may connect to system 130, data sources 110, and/or client device 160 directly via a direct or private network connection (not shown). In some embodiments, third party system 165 may include an Application Program Interface (API) or other data interface to allow one or more computer systems to communicate directly with one or more software applications executed by processors of third party system 165.

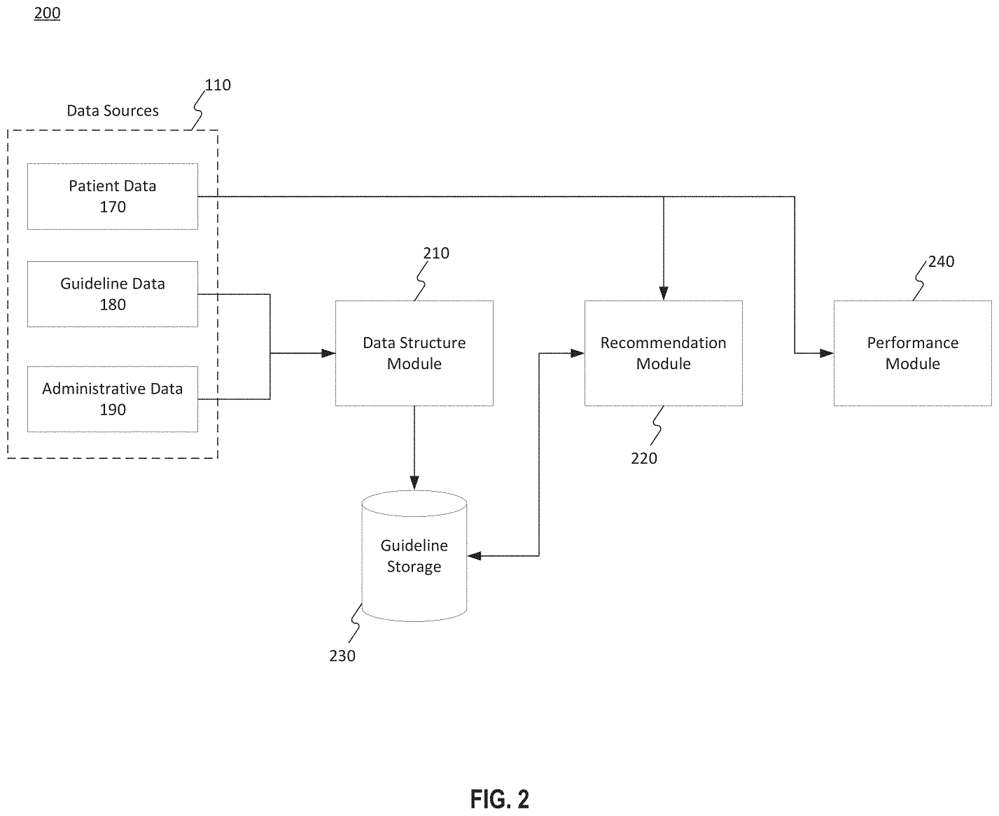

FIG. 2 illustrates system 200, which is an exemplary embodiment of system 130 for implementing embodiments consistent with the present disclosure. System 200 may be implemented as part of system 130 (FIG. 1). For example, system 200 be a component or process of processor 140. In some embodiments, system 130 may parse one or more decision trees received from data sources 110 to identify relationships between clinical attributes and treatments. System 130 may also identify negative relationships between clinical attributes and treatments, thereby obviating a practitioner's need to evaluate a decision point that does not apply to a particular patient.

Data structure module 210 may be configured to receive guideline data 180 and administrative data 190 from data sources 110, e.g., via a data interface. Guideline data 180 and administrative data 190 may be received from one or more local or remote databases. In some embodiments, data structure module 210 may be configured to receive structured input associated with unstructured compendium records. For example, a compendium may store guidelines by drug name. For each drug, the compendium may include free-form block text describing decisions required to reach a treatment recommendation.

Data structure module 210 may receive input indicative of pathways and/or guidelines defined in the compendium and store these pathways and/or guidelines in a structured or unstructured database, e.g., guideline storage 230. For example, a data structure may be generated for each drug, pathway, and/or guideline stored in the compendium. The data structure may be configured to relate a treatment regimen to one or more guideline concordant clinical attributes. In some embodiments, the treatment regimen may be related to one or more indications, where an indication includes one or more patient attributes. For example, a treatment regimen may be guideline concordant if the regimen is indicated for the patient receiving the regimen, i.e., if the patient presents the patient attributes of an indication associated with the treatment regimen.

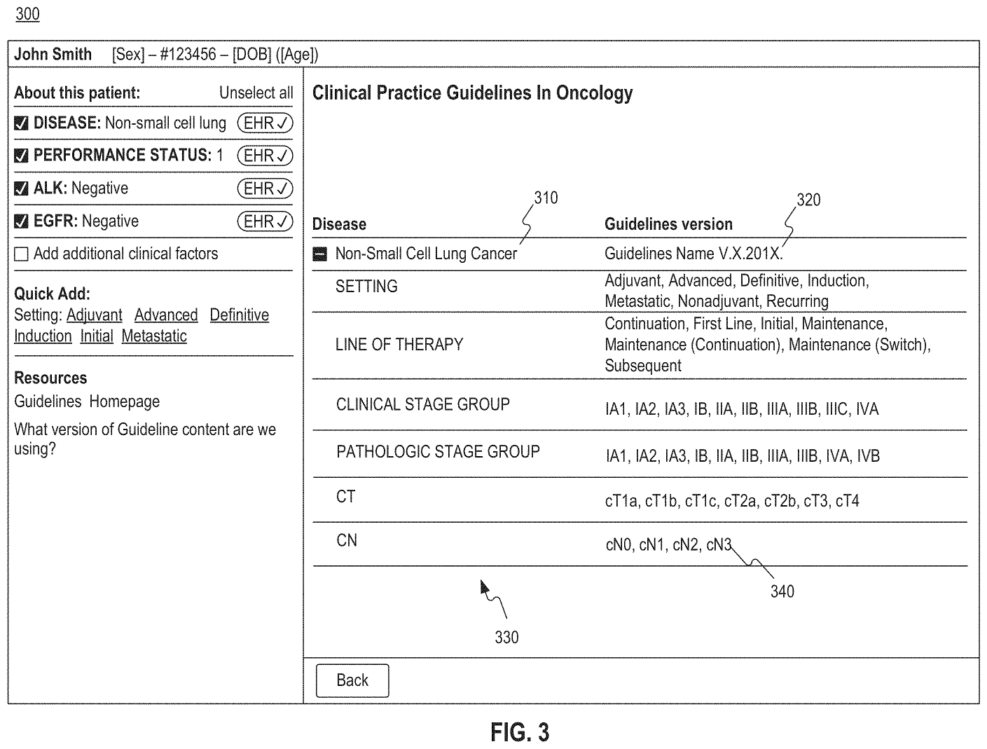

Recommendation module 220 may be configured to receive input from data sources 110 and from client device 160. For example, a user may input data via a graphical user interface (GUI) displayed by client device 160. Exemplary GUIs displayed by client device 160 are described in further detail below with reference to FIGS. 3, 4A-4D, and 5.

Recommendation module 220 may receive input of a search term or of one or more clinical attributes associated with a patient. In some embodiments, the search term may be a drug name, an indication, or a clinical attribute. Recommendation module 220 may query data storage 230 to identify one or more records containing the search term. In some embodiments, the recommendation module 220 may receive, associated with the search, patient data 170. The patient data 170 may be associated, for example, with a particular patient and may include one or more clinical attributes of the patient. Recommendation module 220 may generate a query of the data structure stored by data storage 230 in which the search results are filtered by indication and/or clinical attribute.

In other embodiments, recommendation module 220 may search or filter guideline storage 230 to identify one or more regimens that are concordant for a patient, based on the patient's clinical attributes. For example, recommendation module 220 may filter guideline storage 230 by one or more clinical attributes. In some embodiments, recommendation module 220 may be further configured to filter search results for a drug name by one or more clinical attributes.

Recommendation module 220 may execute the generated query and return a list of one or more regimens associated with the search term and/or clinical attributes. In some embodiments, the list may be ranked by relevance. For example, a relevance score for each regimen may be determined based on a comparison of the search term to a drug name, indication, or clinical attribute of the regimen. Thus, the most relevant regimens or results may be displayed at the top of the list. In other embodiments, search results may be listed with treatments preferred by a hospital or insurance provider listed first.

Recommendation module 220 may be configured to receive input, via client device 160, indicative of a selection of a regimen returned by the search. Responsive to the selection, recommendation module 220 may generate a GUI configured to display one or more concordant indications associated with the selected regimen. Each concordant indication may include a set of clinical attributes that a patient should have in order for the administered regimen to be guideline concordant.

Responsive to a selection of a concordant indication, in some embodiments, recommendation module 220 may prompt the user to enter additional detail associated with the regimen. For example, the user may be prompted to enter a dosage, frequency, route, start date of the regimen, end date of the regimen, and the like. In some embodiments, the user may select a regimen for a patient who does not present clinical attributes associated with a concordant information. If the user selects a non-concordant regimen, the user may be prompted to enter a reason for selecting a non-concordant regimen.

In some embodiments, recommendation module 220 may receive the regimen selection, indication selection, and regimen details and update an EHR associated with the patient with the received information. The EHR may be stored in an EHR database, e.g., database 150, or a remote database. In some embodiments, some or all of the received data may be stored in a reporting database.

In some embodiments, a performance module 240 may receive, from an EHR database, e.g., database 150, a reporting database, and/or an EHR database, aggregated patient data indicating patient regimens and outcomes. For example, performance module 240 may be configured to generate one or more reports comparing outcomes of concordant versus non-concordant patients. Thus, the system 200 may enable a hospital or other patient service provider to track concordance and/or identify trends in patient outcomes thereby leveraging the data generated via recommendation module 220.

FIGS. 3, 4A-4D, and 5 illustrate exemplary graphical user interfaces (GUIs) consistent with disclosed embodiments. The GUIs of FIGS. 3, 4A-4D, and 5 may be displayed to a user via client device 160, which may be capable of receiving user input via keyboard, microphone, or other I/O device.

As shown in FIG. 3, the user may view the underlying data model, e.g., guideline data stored in guideline database 230, via GUI 300. For example, GUI 300 may display a disease 310 associated with the patient, which may be determined based on the patient's EHR. GUI 300 may also display the guideline version 320. The guidelines indicated via GUI 300 may be e.g., guideline data 180 and may be used by processor 140 to generate outputs including suggested regimens and concordant regimens based on a patient's clinical attributes.

In some embodiments, the user may view, via GUI 300, a list of guideline parameters 330. Guideline parameters 330 may be, for example, column names and allowed values associated with guideline database 230. For example, each record of a regimen in database 230 may be stored with each of the parameters 330 having a value from each listing 340 of available values for the parameter. GUI 300 may further assist the user by indicating fields, e.g., parameters 330, by which the user may filter database 230 to identify regimens.

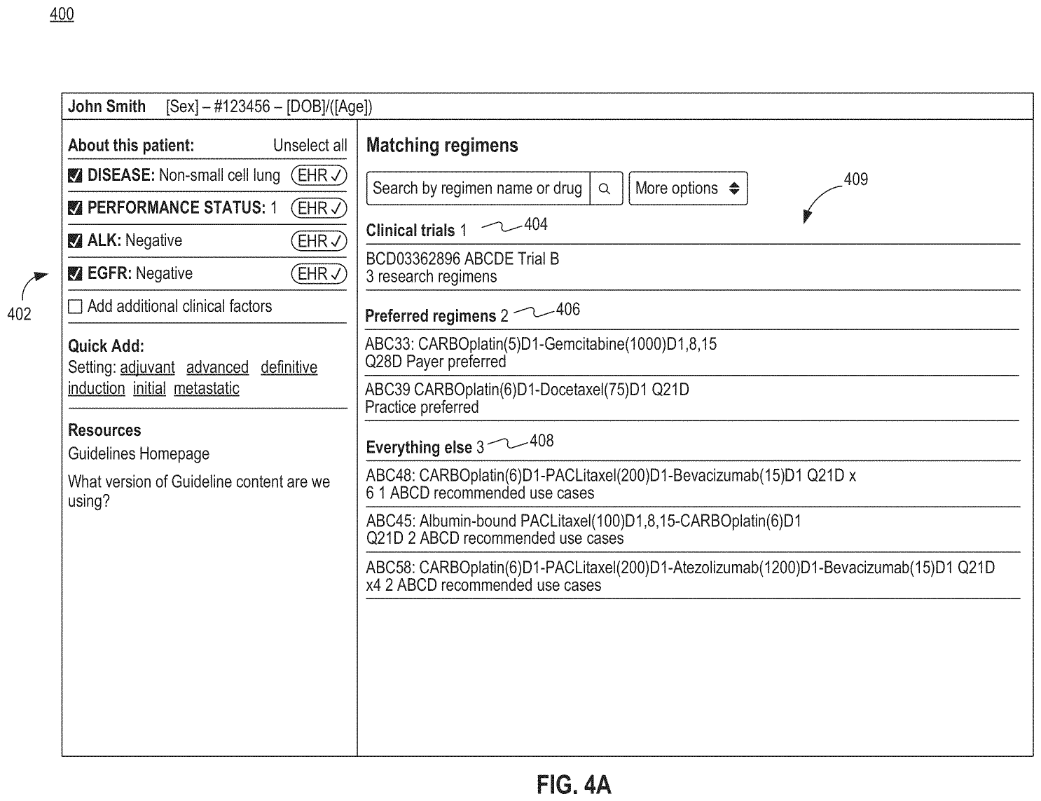

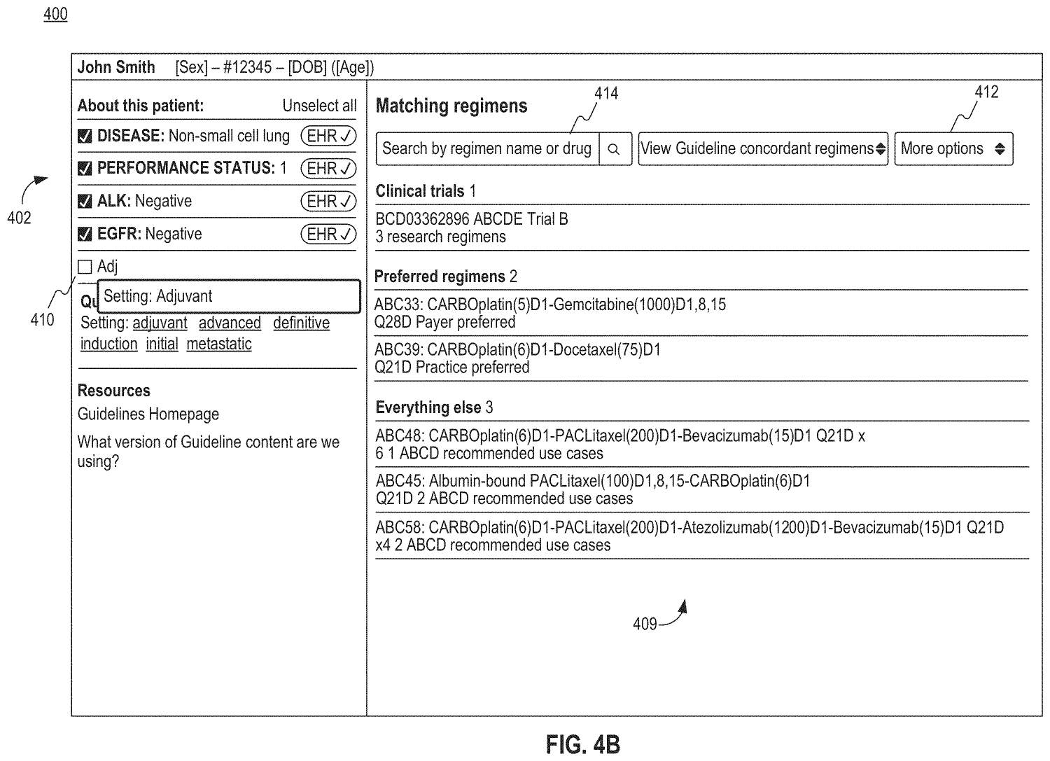

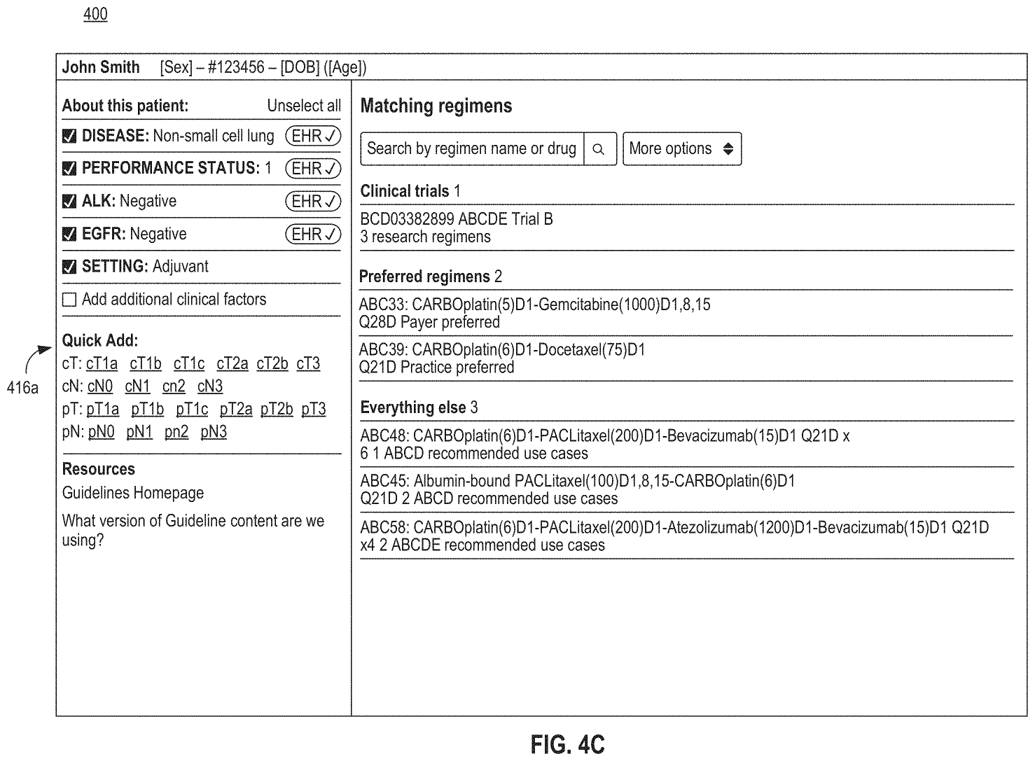

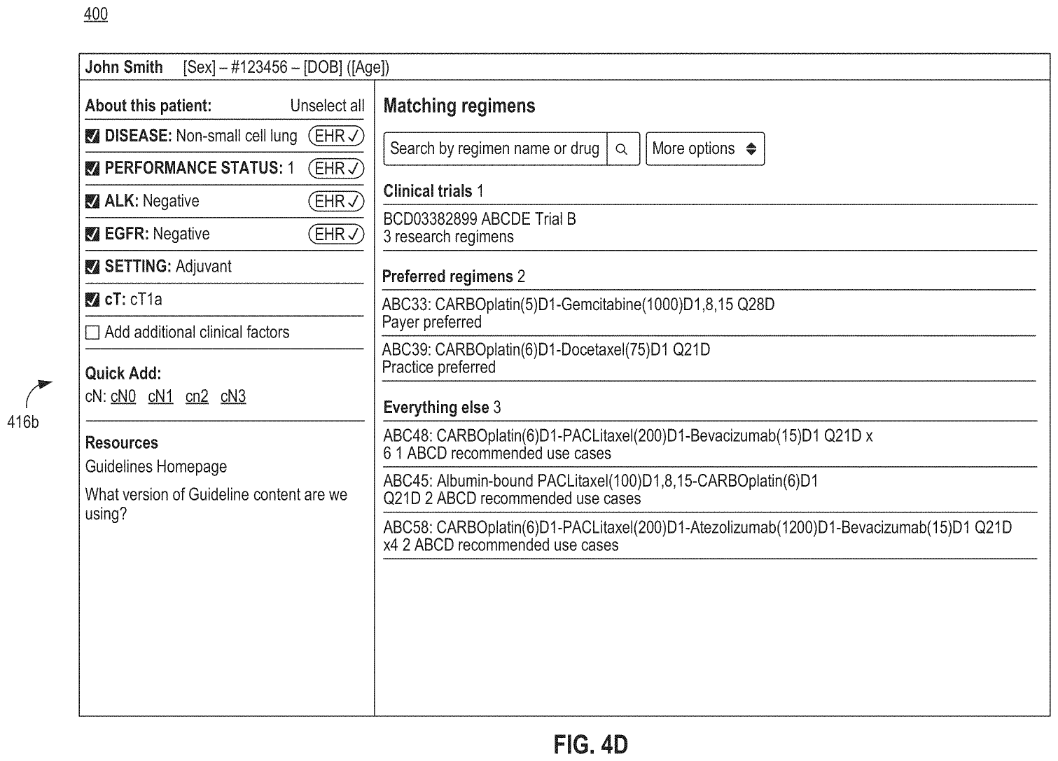

In some embodiments, a user may select a regimen to add to a patient's EHR as described with reference to FIGS. 4A-4D.

For example, as shown in FIG. 4A, a GUI 400 may display one or more regimens determined by recommendation module 220. For example, recommendation module 220 may receive patient data 170 as well as data from guideline storage 230. Recommendation module 220 may generate a list 402 of one or more patient attributes based on patient data 170. For example, each clinical attribute of list 402 may be pulled from data in a patient's EHR. Recommendation module 220 may then query guideline storage 230 using these attributes to filter guideline concordant regimens for the patient.

GUI 400 may be configured to display a list of search results 409. The search results may be generated based on a query of one or more regimen databases as described above with reference to recommendation module 220. The regimen database may be, e.g., database 150 of system 100, or a remote database, e.g., a database associated with guideline data 180. For example, recommendation module 220 may generate a query to retrieve concordant regimens from guideline database 230 by filtering database records based on the values of filters 702.

In some embodiments, data structure module 210 may receive administrative data 190 indicative of practice or payer preferred treatments, and/or clinical trial regimens. For example, data structure module 210 may be configured to receive administrative data 190, which may include information associated with one or more clinical trials. Data structure module 210 may be configured to generate structured database records for each clinical trial, such that the available clinical trials are searchable in data storage 230. In some embodiments, data structure module 210 may be configured to generate unstructured database records, depending on the system architecture and requirements for the particular implementation. In some embodiments, the system may receive, from recommendation module 220, administrative data 190 indicative of preferred treatments, e.g., treatments preferred by an insurance company, or of potential clinical trials. This data may also be displayed to the user via GUI 400 as part of the list of concordant regimens. Thus, GUI 400 may be used to indicate to the user, e.g., the physician, preferred treatments or trials available for a patient having a particular set of clinical attributes. For example, GUI 400 may be configured to display, in the list of results 409, a list of concordant clinical trials 404, preferred regimens 406, and/or other concordant regimens 408 based on administrative data 190. In some embodiments, the system may employ one or more machine learning techniques to dynamically train, update, and improve algorithms for predicting a practice's or payer's preferences. For example, a practice's preferred treatments may be updated dynamically, based on monitoring and tracking regimens that are commonly selected by a practice to treat a patient with particular characteristics suffering from a particular disease. Similarly, a payer's preferred treatments may be updated dynamically, based on, for example, monitoring and tracking selected regimens that receive prior authorization, treatments that are denied prior authorization, and/or biosimilar substitutions that are made for a patient with particular characteristics and suffering from a particular disease.

In some embodiments, the system may be further configured to retrieve trial regimen data from a database, e.g., data source 110. The system may generate GUI 400 to display one or more trial details in addition to the trial regimen. Trial details may indicate, for example, inclusion characteristics and exclusion characteristics to aid the user in determining whether the patient is eligible for the trial.

Further, in some embodiments, preferences of a payer or a practice can be preloaded and only those regimens preferred by the payer and/or practice may be displayed.

In some embodiments, via GUI 400, a user may select from a drop-down menu of “More options” 412. For example, the user may select to further filter the results by guideline concordant regimens, or may select to view unavailable guideline templates. Thus, the user may view regimens matching a search term input via search field 414, rather than the list of attributes 402 pulled from the patient's EHR. In this embodiment, recommendation module 220 may query guideline database 230 using the search term received via search field 414 and return one or more regimens associated with the search term, e.g., where the regimen record in guideline database 230 matches or contains the search term.

For example, if a user selects to view unavailable regimens, the user may select a regimen displayed via GUI 400 even if the patient does not have clinical attributes of any concordant indication. In this case, the user may select a reason for administering a non-concordant treatment regimen. The reason may be selected from a drop-down menu. In other embodiments, a GUI may include a text field configured to receive free form input. In some embodiments, the user must select a reason for administering a non-concordant treatment regimen prior to continuing the workflow.

In some embodiments, once the user selects a regimen or trial regimen from list 409, e.g., via a touchscreen or I/O device of client device 160, the system may generate a GUI configured to display a list of concordant indications associated with the selected regimen. Each concordant indication may include a set of clinical attributes that should be present in the patient for the regimen to be concordant and may list and/or otherwise indicate attributes of the patient based on structured or unstructured data pulled from the patient's EHR. For example, a concordant indication may be associated with the clinical attributes of: a cancer type, e.g., non-small cell lung cancer (NSCLC), tumor, node, and metastasis (TNM) staging information, e.g., T2aN0M0 or T2bN0M0, therapy type, e.g., adjuvant, and a residual tumor classification, e.g., RO. Other clinical attributes may be associated with an indication.

In some embodiments, GUI 400 may be configured to receive a selection of a regimen from the list of search results 409, and, responsive to the selection, the GUI may display a pathway associated with the selected regimen. In other embodiments, a GUI may be configured to display an indication of payer or provider preferred regimens or clinical trial regimens associated with the received search term.

As shown in FIG. 4C, in some embodiments, GUI 400 may be configured to display a list 416a of “Quick Add” attributes. The list 416a may be generated dynamically based on the patient's clinical attributes and on the guidelines. For example, the additional “Quick Add” attributes may be determined based on criteria for pathways matching the patient's clinical attributes. The user may select from the “Quick Add” list 416a to further narrow the search criteria applied to guideline database 230. Other exemplary filters, not shown, may include bio-markers, line of therapy, pathologic stage group, and the like. Available filters may be based on, for example, the parameters of the selected guidelines, as shown in FIG. 3.

FIG. 4D displays another view of GUI 400 in which a user has selected a cT value of cT1a from the Quick Add list 416a. Recommendation module 220 may then dynamically retrieve available attributes by which to further filter available regimens and display those attributes as Quick Add list 416b. For example, by selecting cT1a, the user may then only select further filters from the list of cN values, for which concordant regimens are available. In another embodiment, the filters displayed in the Quick Add lists 416a and 416b may be determined based on whether the presence of a certain attribute eliminates the possibility of a patient possessing another attribute.

In another embodiment, GUI 400 may be configured to generate, based on structured and/or unstructured data generated by data structure module 210, a decision tree associated with the search term input via field 414, or the filter values 402 pulled from the patient's EHR. In some embodiments, the displayed decision tree may be dynamic, such that a user may select from one or more links to view pathways of the generated decision tree. In some embodiments, the displayed decision tree may be generated, in part, based on patient data. For example, the displayed pathways may be selected based on data retrieved from the patient's EHR. If the patient's medical history indicates the patient's NSCLC is in a pathologic stage pT2a, N0 or pT2b, N0, GUI 640 may display the associated decision tree.

In some embodiments, after a user selects a regimen, the system may generate a GUI configured to receive additional detail regarding the selected regimen. For example, the user may input information including, for example, a treatment start date, a treatment end date, a line of treatment, a treatment goal, a treatment plan provider, and/or a treatment department, respectively. In some embodiments, some or all of the additional information may be optional. Additional details regarding the patient may be retrieved from guideline storage 230 or from another database storing regimen information. In some embodiments, additional information, e.g., dosage information or demographics may be retrieved from the patient's EHR.

After inputting and reviewing the information input, a user may add the treatment plan to the patient's EHR. The system may update and/or save the patient EHR with the added treatment plan to an EHR database. In some embodiments, all or part of the information may be saved to a reporting database. After accepting the treatment plan, a GUI of system 130 may display a summary of the received treatment plan data. For example, treatment plan information may be accessed at any time by the user through the patient's EHR.

As previously described, the system may save treatment plan data received via GUI 400 to a reporting database. The system may be used, for example, by a hospital administrator to generate one or more reports associated with aggregate guideline concordance data.

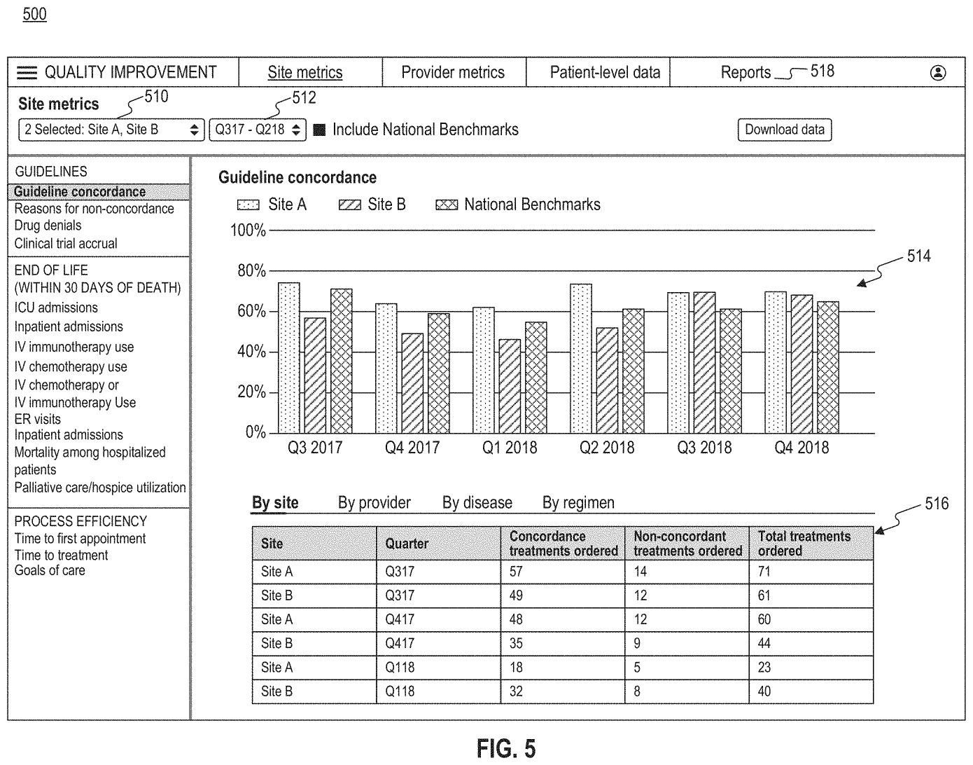

FIG. 5 illustrates a GUI 500 displaying an exemplary guideline concordance report. Via GUI 500 a user may access an EHR database and/or reporting database to analyze aggregate data. For example, GUI 500 may be configured to receive one or more parameters, e.g., a site 510 and a date range 512. In other embodiments, GUI 500 may be configured to receive any number of parameters, e.g., a date, a regimen, a medical provider, etc. In other embodiments, GUI 500 may be configured to receive a structured database query. In some embodiments, the database query may be directed to one or more unstructured databases, or a combination of structured and unstructured databases.

GUI 500 may include a graph view 514 and a tabular view 516. Performance module 240 may be configured to receive, via GUI 500, the one or more parameters and generate a query of the reporting database and/or EHR database. Based on the query results, performance module 240 may generate a visual representation, e.g., graph 514, of the returned data. Performance module 240 may also generate a table 516, which may be filtered by site, provider, disease, or regimen. In yet another embodiment, performance module 240 may query one or more databases storing national standard data and display, via GUI 500, benchmark data.

GUI 500 may enable administrators and providers to view summaries and trends of concordance data, which may be useful in guiding treatment decisions or identifying preferred treatments. GUI 500 may also be configured to display data associated with administered non-concordant regimens and/or patient outcomes. Thus, a provider or administrator may be better able to leverage concordance data to improve patient outcomes and patient care. In some embodiments, the system may be configured to generate and output one or more reports on payer responses to a prior authorization request: e.g., what regimens were approved, denied, and/or substituted. Such reports may be generated in response to selection of reports tab 518.

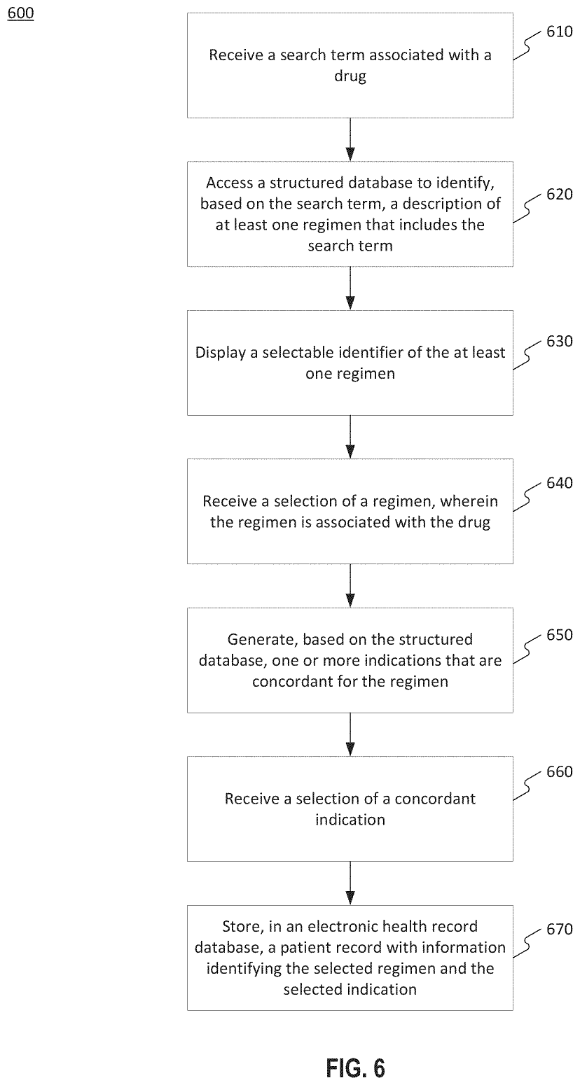

FIG. 6 illustrates an exemplary process 600 for providing guideline concordance. Process 600 may be implemented, for example, a processor 140 of system 100, shown in FIG. 1.

At step 610, the system may receive a search term associated with a drug. For example, a user may input a search term via GUI 400, or system 130 may pull patient information from a patient's EHR and perform a search using the patient's clinical attributes. In other embodiments, the user may search by indication and/or clinical attribute to identify regimens associated with the input indication and/or clinical attribute.

At step 620, processor 140 may access a structured or unstructured database to identify, based on the search term, a description of at least one regimen that includes the search term. For example, based on the search term, processor 140 may query guideline storage 230 to identify at least one regimen containing or matching the search term. In some embodiments, the query may identify at least one regimen containing a partial match of the search term. In some embodiments, processor 140 may identify a regimen based on whether the regimen name, description, or other associated information contain at least a partial match of the search term.

As previously described, the database may be a structured database generated by data structure module 210 and may be based on a guideline compendium containing unstructured data. In some embodiments, the database may be an unstructured database. In some embodiments, the guideline compendium may include a number of decision trees. Data structure module 210 may generate structured or unstructured guideline records automatically or may be configured to receive input from an administrator. Data structure module 210 may be configured to identify and store relationships between clinical attributes and regimens, thereby enabling a physician to identify appropriate treatment regimens, without needing to navigate through an ordered list of decisions.

At step 630, processor 140 may display, via a graphical user interface, a selectable identifier of the at least one regimen. For example, processor 140 may generate a GUI, as shown in FIGS. 4A-4D. Processor 140 may transmit the search results to client device 160, thereby causing client device 160 to display the GUI. In embodiments in which the search term is an indication or clinical attribute, the GUI may display a list of regimens that are associated with the indication and/or clinical attribute.

As previously described, processor 140 may determine a relevance score associated with each record based on, for example, a number of times the search term appears in the record, or whether one or more patient attributes match the clinical attributes associated with the record. The GUI may be configured to display a ranked list of search results, where the most relevant results are listed first. In another embodiment, the GUI may display a predetermined number of results, e.g., the top 20 results.

At step 640, processor 140 may receive a selection of a regimen. For example, the user may click on or hover over a regimen on a display of client device 160. The selection may be transmitted to processing device 140 via network 120. In some embodiments, processor 140 may be a processor of client device 160. The selection may be received, for example, as a result of the user clicking on the regimen with a cursor or hovering over the regimen. In some embodiments, the GUI may include one or more of a radio button, a link, or a checkbox configured to receive a selection from the user.

At step 650, responsive to the user's selection of a regimen at step 640, processor 140 may generate, based on the structured or unstructured database, one or more indications that are concordant for the regimen. For example, recommendation module 220 may generate a query of data storage 230 configured to retrieve data associated with the selected regimen. Data associated with the regimen may include one or more concordant indications and/or clinical attributes, where an indication may be a set of one or more clinical attributes.

At step 660, processor 140 may receive a selection of a concordant indication. For example, in some embodiments, the user may select a concordant indication with which the patient shares clinical attributes. As previously described, in some embodiments, the patient may not match any concordant indications. In this case, the user may select to proceed with the non-concordant regimen. In some embodiments, the system may require a user to input a reason for selecting a non-concordant treatment for the patient. The input may be received, for example, as free form text, or may be a prepopulated reason selected from a drop-down menu.

In other embodiments, the retrieved search results may be filtered based on the patient's clinical attributes. In such embodiments, the displayed list of regimens may all be guideline concordant. In some embodiments, e.g., via GUI 400, a user may select whether to view only concordant regimens or view non-concordant regimens.

At step 670, processor 140 may store, in an electronic health record database, a patient record with information identifying the selected regimen and the selected indication. For example, processor 140 may update a patient EHR in an EHR database. In some embodiments, the input received in process 600 may also be stored in a reporting database.

Attention is now directed to the electronic prior authorization functionality of the disclosed embodiments. While or after identifying candidate treatment regimens using, for example, the guideline concordance techniques previously discussed, caregivers seeking to implement a treatment regimen often seek prior insurance authorization to ensure that the treatment will be paid by the insurance provider. The disclosed embodiments integrate, within a practice management system or contained in a separate connected system, a workflow for facilitating automated data collection, electronic message generation, and transmission to seek pre-approval from an insurance provider or related system, without the need for entering information into multiple different systems. The disclosed embodiments also provide dynamic graphical user interfaces that facilitate the pre-approval process to obtain insurance provider authorization in near-real time and at the point of care.

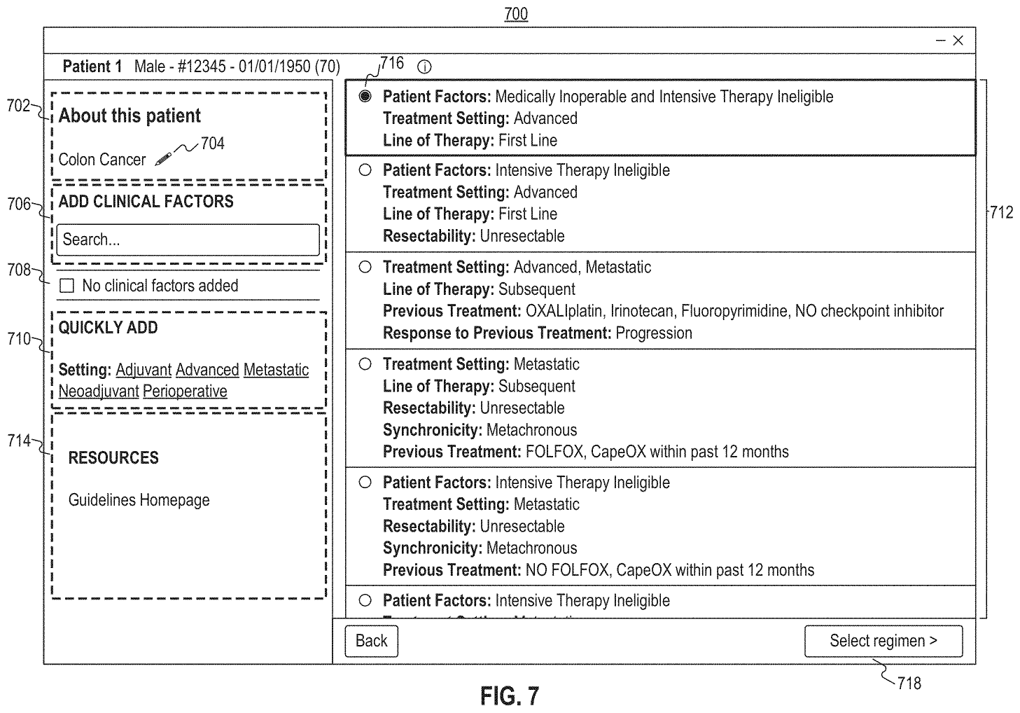

FIGS. 7-12 illustrate graphical user interfaces for obtaining automatic approvals from insurance providers, or from entities acting on their behalves, for treatment regimens selected by caregivers. As described in further detail below, the automatic approvals may be obtained using a single round of communications between system 130 and a third party system 165 operated by the insurance provider or on their behalves. The disclosed embodiments improve upon traditional systems by automatically compiling information about a patient, applying logic, rules, and guidelines to automatically generate proposed supplemental information to add to the patient's data, automatically applying rules, logic, and guidelines to identify and present selectable treatment regimen options, and automated generation of one or more electronic messages that pass through an Application Programming Interface (API) to the third party system, to receive approvals via the API in near-real time.

FIG. 7 is an illustration of an exemplary regimen selection GUI 700 consistent with the present disclosure. In some embodiments, processor 140 may generate the regimen selection GUI based on information retrieved from one or more data sources 110. For example, processor 140 may retrieve patient data 170 to populate fields in the GUI for a patient name, identification number, date of birth, age, sex, and any additional relevant information stored as patient data 170, as previously discussed.

In some embodiments, regimen selection GUI 700 may automatically provide information about the patient's current condition, in an “About this patient” region 702. In the example in FIG. 7, the patient has been previously diagnosed with colon cancer, and this condition may be retrieved from patient data 170 by processor 140 and automatically displayed. Regimen selection GUI 700 may include one or more interactive elements for modifying the patient condition, such an “Edit” button or a selectable pencil icon 704, as shown. Regimen selection GUI 700 may also provide one or more interfaces for processor 140 to receive additional information about the patient, such as an input box 706 for adding clinical factors/attributes, or a selectable element 708 for receiving an indication that there are no other clinical factors to add.

In some embodiments, processor 140 may automatically generate one or more interactive elements for adding clinical factors, or clinical attributes, to patient data 170, such as those shown in the “Quickly Add” region 710, for adding clinical attributes as previously discussed with respect to FIG. 4C. In the example shown in FIG. 7, processor 140 automatically presents selectable elements in region 710 for adding “Adjuvant,” “Advanced,” “Metastatic,” “Neoadjuvant,” and “Perioperative.” Processor 140 may automatically identify a set or subset of clinical factors to add based on the patient condition stored in patient data 170. For example, processor 140 may retrieve the patient's condition, and query guideline data 180 to identify one or more clinical factors in the guideline data 180 decision trees that have not yet been identified with respect to the patient's condition. For example, if the patient has been diagnosed with cancer, processor 140 may retrieve a cancer stage indication from patient data 170, and may automatically identify and display treatment regimens associated with the identified cancer stage, as identified in guideline data 180. By automatically identifying potential clinical factors associated with the patient's condition, the disclosed embodiments can improve the consistency and quality, while minimizing the number of communications required to obtain treatment authorization and payment pre-approval from insurers. Additional details are discussed above with respect to FIGS. 4C and 4D.

In some embodiments, processor 140 may automatically identify a plurality of treatment regimens based on the patient data 170, including a treatment plan and one or more medications. The identified regimens may be displayed in a treatment regimen list 712 In some embodiments, recommendation module 220 may identify candidate treatment regimens, as previously described with respect to recommendation module 220 and FIG. 6. The identified treatment regimens may correspond to treatment regimens set forth in the guidelines applied for the patient. Processor 140 may select the treatment regimens to display based on the patient data 170, clinical factors identified in regimen selection GUI 700, and any other resource databases or preprogrammed decision trees. Processor 140 may also provide a Resources region 714 identifying one or more resources applied to select the treatment regimens. As shown in in region 714 of FIG. 7, processor 140 has applied the relevant Guidelines and UpToDate resources, and presents at least six treatment regimens in a scrollable portion of GUI 700.

In some embodiments, processor 140 may provide detail information for each of the treatment regimens presented in treatment regimen list 712. Displayed details may be extracted from data sources 110 and can include, for example, Patient Factors associated with the treatment regimen, treatment setting, line of therapy, resectability (such as the ability to remove a mass of tissue such as a tumor), synchronicity, a description of previous treatment, and the patient's response to previous treatment (including progression or regression).

In some embodiments, processor 140 may identify all potential treatment regimens for a particular patient as discussed above, but display only a subset of the identified treatment regimens in list 712 based on information received from a third party insurance provider system identifying preferred treatment regimens. In some embodiments, processor 140 may display all identified treatment regimens, and provide a graphic visual indication (not shown) identifying an insurance provider's preferred treatment regimens, such as by providing an icon, color coding, or shading to distinguish preferred treatment regimens in the complete list 712.

GUI 700 may allow a caregiver to select a treatment regimen by selecting a particular treatment regimen from a selectable graphical element next to each treatment regimen. In the example shown, a radio button 716 is provided to the side of each treatment regimen in list 712. GUI 700 may prevent the selection of multiple treatment regimens by allowing selection of only a single treatment regimen radio button. Once processor 140 identifies selection of a “Select Regimen” button 718, processor 140 may generate an electronic message for seeking approval from the insurance provider system. In some embodiments, GUI 700 may allow a caregiver to input a treatment regimen that is not automatically displayed in GUI 700, or allow selection of a potential treatment regimen that is displayed yet identified as a non-recommended regimen due to patient data 170 or guideline data 180. In this situation, GUI 700 may also provide an interactive user interface element to allow a caregiver to provide comments or reasons for explaining why the caregiver is selecting a non-recommended regimen, for consideration and analysis by the insurance provider system.

In some embodiments, processor 140 may generate an electronic message that includes patient data 170 or a selected subset thereof, and the selected treatment regimen. The electronic message may include information for the insurance provider or related system to automatically approve the selected treatment. Processor 140 may automatically determine the subset of patient data 170 to provide based on predetermined requirements of the insurance provider system, the applied guidelines and predetermined data fields set forth in the guidelines for the selected treatment regimen, and any other predetermined settings for the caregiver, guidelines, or insurance provider. Processor 140 may generate the electronic message in a format suitable for transmission via an application programming interface (API) to a third party system 165 such as an insurance provider system. The electronic message may be provided to the insurance provider system via the API, and the insurance provider system may employ one or more rule sets to authorize, reject, request additional information, or recommend a biosimilar with respect to the selected treatment regimen, as discussed in further detail below.

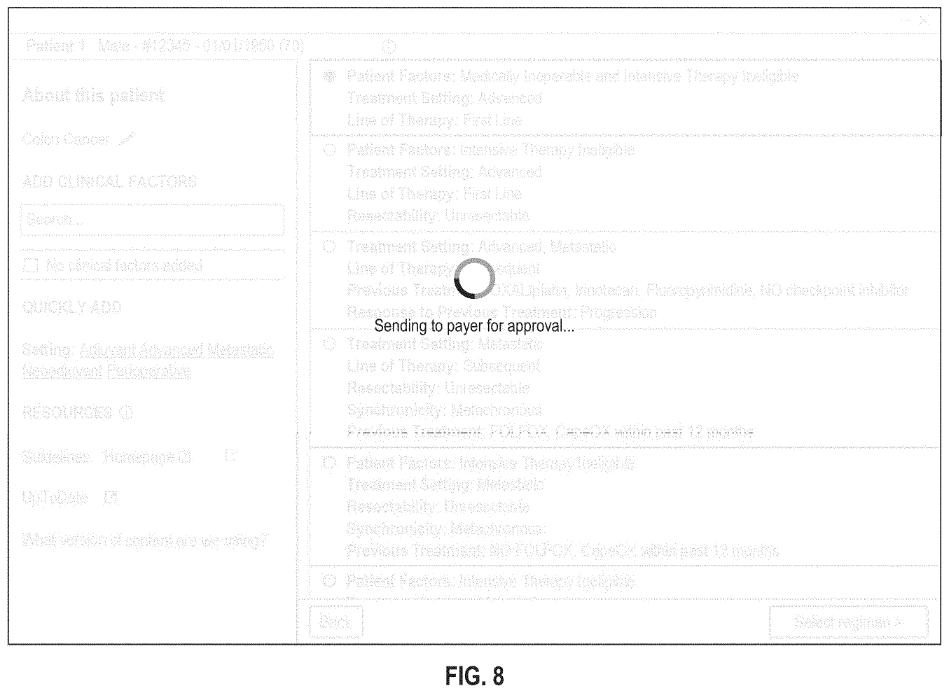

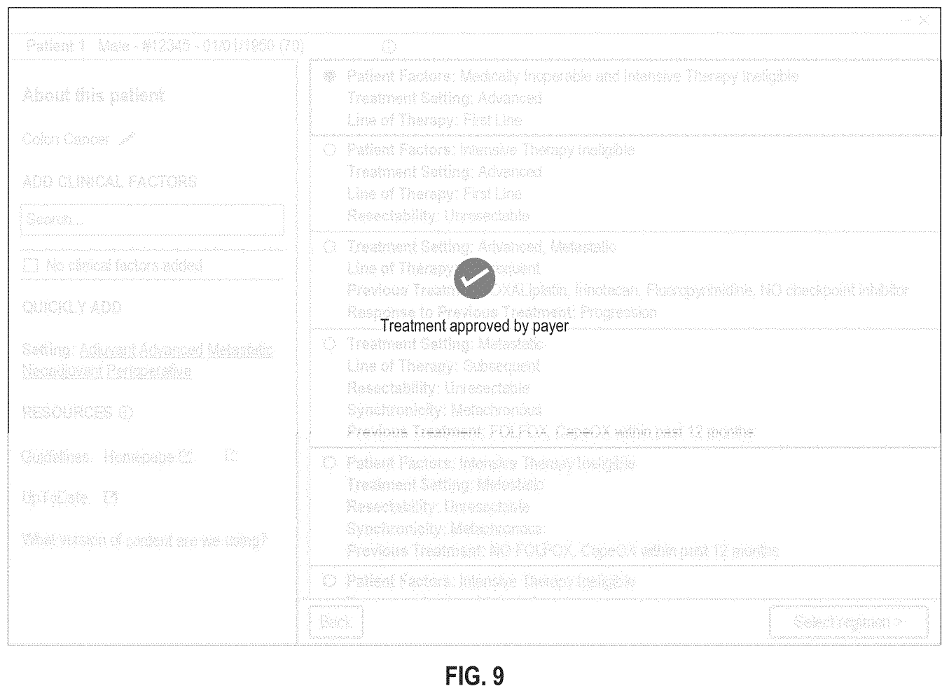

As shown in the graphical user interface depicted in FIG. 8, processor 140 may generate for display a graphical user interface for providing a real-time status for the transmission of the electronic message to the insurance provider or related system. As shown in FIG. 8, a graphical user interface is generated and displayed to indicate that the electronic message is being sent to a payer (e.g., the insurance provider) for approval. As shown in the graphical user interface depicted in FIG. 9, processor 140 may generate for display a graphical user interface indicating that the selected treatment regimen has been approved by the insurance provider system. The received indication from the insurance provider system may include a responsive status generated by the insurance provider system, based on an application of one or more rule sets implemented by the insurance provider system for authorizing, rejecting, requesting additional information, or recommending a biosimilar with respect to the selected treatment regimen. The third party system 165 may provide one or more predetermined automated responses stored in a database associated with the third party system. In some embodiments, the automated responses may be used to update an insurance provider's preferences determined and stored by processor 140, to improve the speed and accuracy of future computing and approval process workflows. By implementing the disclosed embodiments, the electronic message may be sent and approval may be received within seconds or minutes, with a single round of communication between the caregiver's system and the insurance provider's system. The disclosed embodiments may therefore conserve computer and communication resources that are often burdened with numerous rounds of data transmission using traditional insurance approval techniques. The disclosed embodiments also improve the caregiver user's experience by involving a single interaction with the graphical user interface, and by providing greater predictability in coverage using predefined guidelines to automatically adjust patient data and the identification of potential treatment regimens.

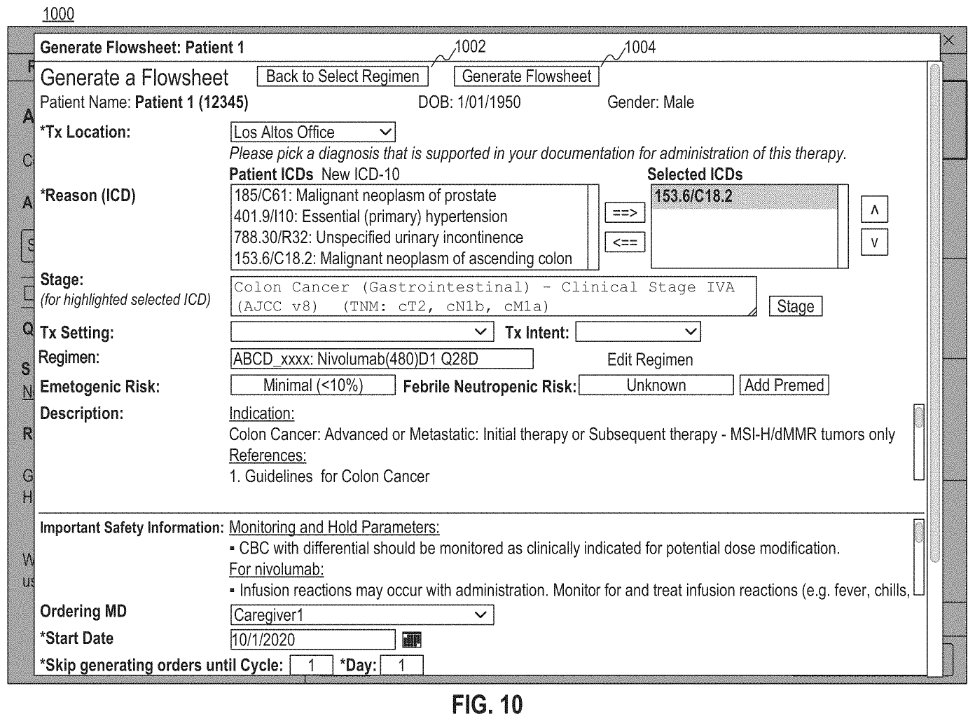

Following the automated response from the insurance provider system for the selected treatment regimen, processor 140 may generate for display a flowsheet generation GUI 1000, such as the example illustrated in FIG. 10. As shown, GUI 1000 may include information automatically populated for the selected treatment based on stored guidelines and patient data. In some embodiments, GUI 1000 may include all fields automatically populated with patient data and treatment details. Certain fields of GUI 1000 may include interactive elements that are selectable or editable by a user such as the caregiver. For example, GUI 1000 may include one or more editable field boxes, dropdown menus, or selectable buttons for editing treatment information for the approved treatment. In some embodiments, processor 140 may automatically restrict the editable fields to those that do not substantively affect the approval status of the treatment regimen. GUI 1000 may include one or more buttons for allowing a user to revert back (“Back to Select Regimen” button 1002) to the regimen selection screen (FIG. 7), or proceed to Generate Flowsheet (“Generate Flowsheet” button 1004) based on the parameters in flowsheet generation GUI 1000.

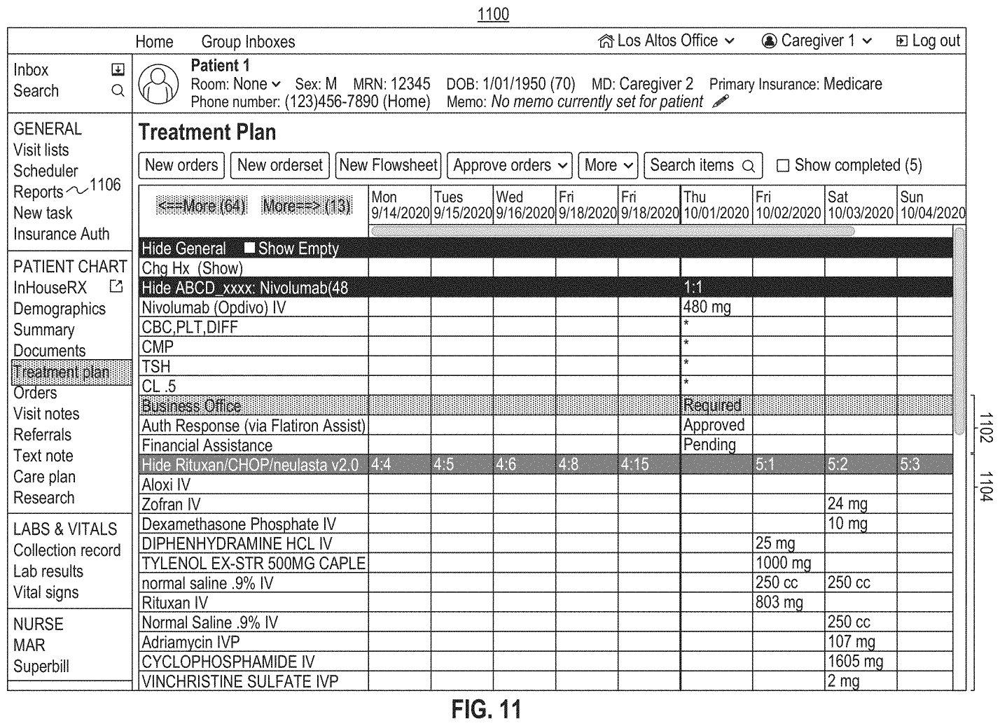

After selection of the “Generate Flowsheet” button 1004 in GUI 1000, processor 140 may generate for display a graphical user interface such as treatment plan GUI 1100, such as the example shown in FIG. 11. As shown, GUI 1100 may provide a flowchart illustrating the selected treatment regimen, as applied for the particular patient based on the patient data 170, guidelines 180, and any relevant administrative data 190. Status portion 1102 of GUI 1100 may be displayed to provide approval statuses based on administrative data 190, indicating the approval statuses that are pending or received for the treatment regimen. GUI 1100 may provide a detailed breakdown table 1104 illustrating tasks and/or medications to administer on predetermined treatment dates.

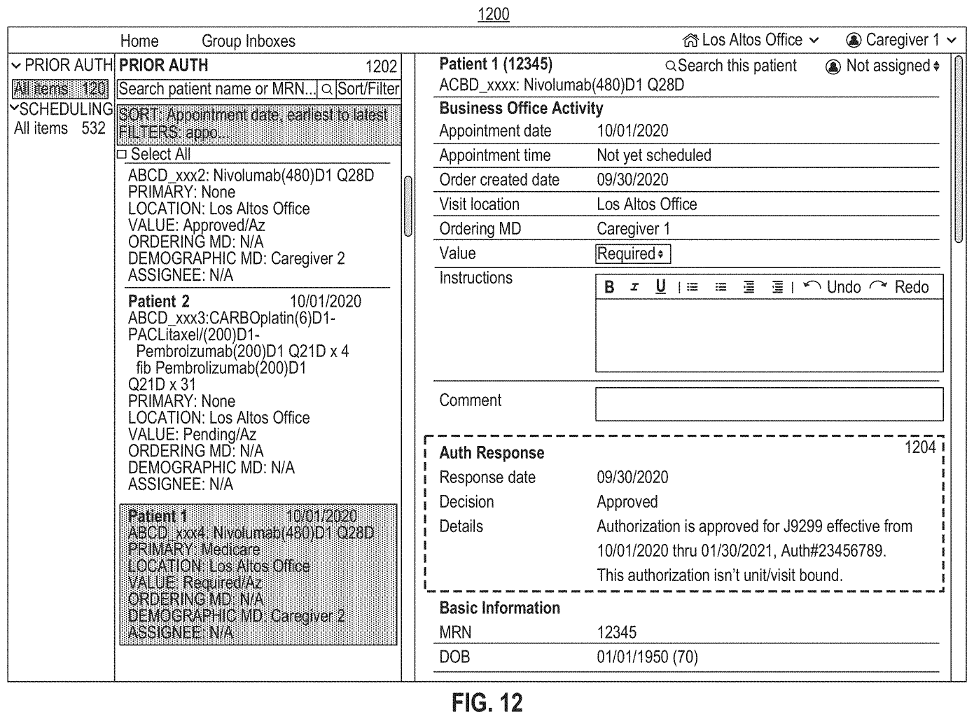

FIG. 12 shows an illustration of prior authorization GUI 1200, including a group messaging inbox that can allow a caregiver to view patients and received electronic messages indicating authorization responses. Received electronic messages may provide information to a caregiver regarding further actions needed, such as a request for a doctor to select a biosimilar, provide an insurance provider system with additional patient information, or other actions that may be required for authorization based on one or more rule sets implemented by a third party system 165 operated by an insurance provider. In some embodiments, GUI 1200 may provide an Auth Response portion 1202 showing a visual indication that a prior authorization was received for a particular patient and regimen, to indicate to the caregiver that no further action is necessary. In some embodiments, when additional information is required the electronic message(s) may include one or more interactive graphical elements, such as a hyperlink or button, which when selected, may cause processor 140 to access a website or other electronic interface to a third party system 165 operated by the insurance provider (not shown), for allowing the caregiver to provide the additional requested information. The electronic interface may provide one or more graphical elements for allowing the caregiver to request authorization following entry of the additional information, so that the patient data 170 and/or another patient record can be updated.

In some embodiments, the electronic message may indicate a request for an update to the selected treatment regimen such as a request from the insurance provider to accept a biosimilar (not shown). One or more rule sets implemented by processor 140 and/or third party system 165 may require manual approval of the biosimilar by a caregiver such as the patient's doctor. The electronic message may therefore include one or more interactive graphical elements for accepting, denying, or providing an alternative response to the requested biosimilar.

As shown in FIG. 12, GUI 1200 may include a scrollable menu of patients, their conditions and treatment summary information, and an adjacent window providing detailed information for a patient selected from the scrollable menu. GUI 1200 may include a portion providing authorization response information populated based on a response received from the insurance provider system. In the example shown, processor 140 has generated GUI 1200 including an “Auth Response” portion 1204 with information about the approval status (“Approved”), the status date (“Sep. 30, 2020”) and details about the approved treatment/medication, an effective date range for the approved treatments, an authorization number, and any associated details received from the insurance provider system.

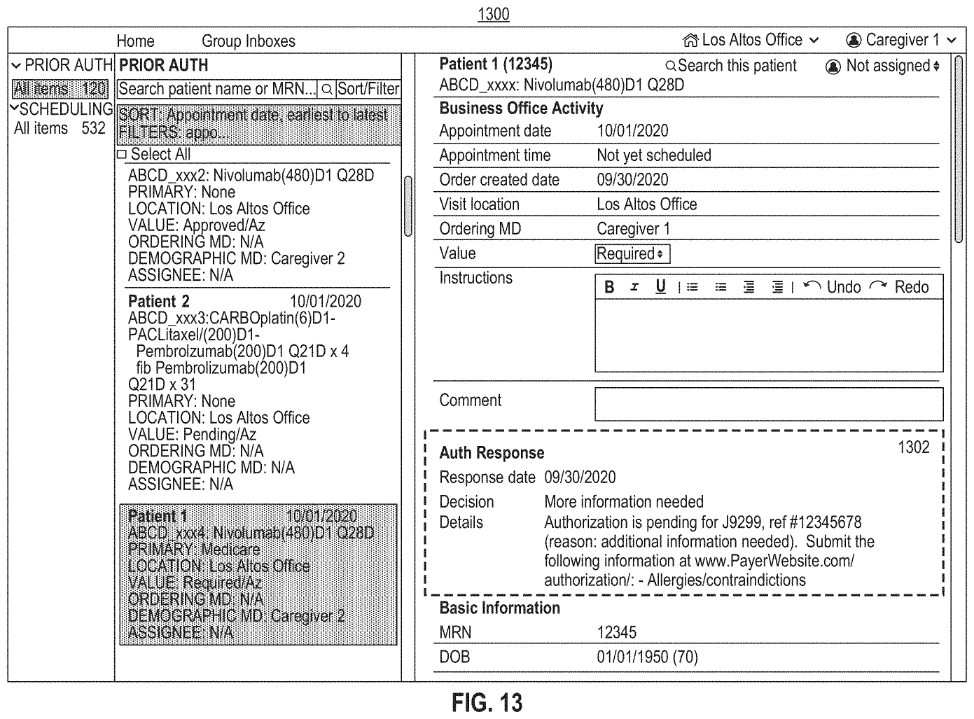

FIG. 13 illustrates a group messaging inbox interface that may allow a caregiver to view patients and received electronic messages indicating authorization responses. In some embodiments, the messaging inbox may be provided in a graphical user interface that may be displayed to the caregiver if third party system 165 is unable to automatically approve the requested treatment in a single communication session. In this situation, the insurance provider may request additional information as a condition for approval of the treatment regimen. Such a situation may arise, for example, if the insurance provider requires confirmation that the requested treatment will not be affected by the patient's allergies or other contraindications. Following the graphical user interface shown in FIG. 8, in which processor 140 is seeking a near-real time approval of the selected treatment regimen, processor may generate for display an alternate prior authorization GUI 1300, as illustrated in FIG. 13. GUI 1300 may have the same general layout and content as prior authorization GUI 1200, except that “Auth Response” portion 1302 shown in FIG. 13 may now display an alternative decision status (“More information needed”) and provide details for the required additional information. GUI 1300 may include one or more interactive elements such as hyperlinks for directly submitting the additional requested information, thereby allowing the caregiver to update the treatment approval request directly from the same system user interface. In some embodiments, one or more of the hyperlinks may connect directly to a website associated with a third party system 165 operated by a payer, such as an insurance provider.

In some embodiments, processor 140 may also generate for display flowsheet generation GUI 1000 (shown in FIG. 10) and a modified version of treatment plan GUI 1100 (show in FIG. 11). With respect to treatment plan GUI 1100, processor 140 may indicate that an “Auth Response” in status portion 1102 is “More info needed” rather than “Approved.”

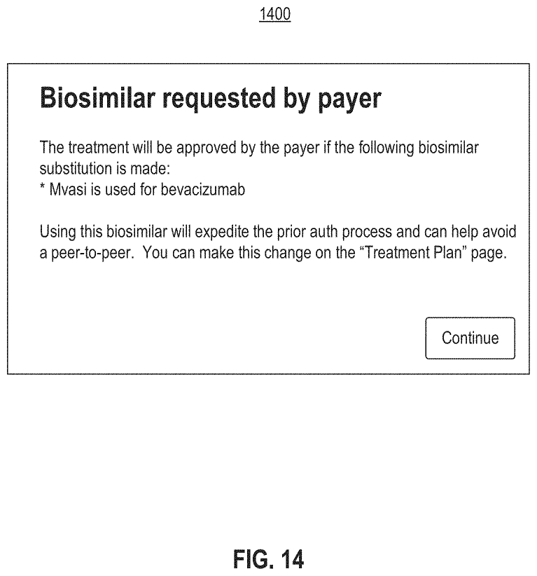

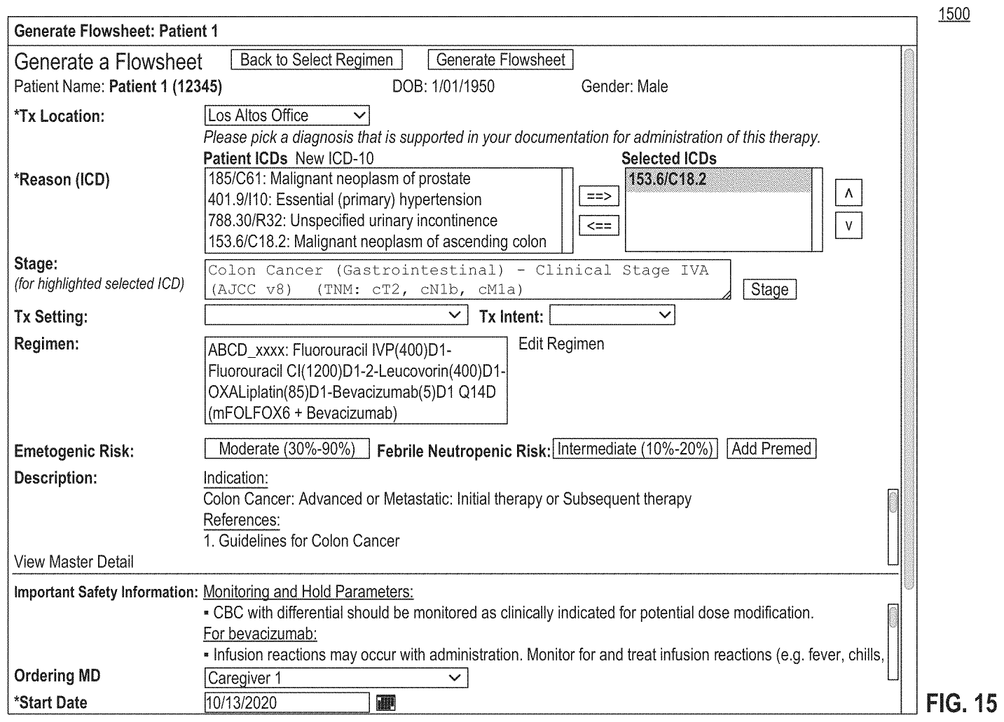

In some embodiments, the insurance provider system may automatically identify an alternative medication or treatment regimen that is expected to provide similar quality, safety, and efficacy as the selected treatment regimen, sometimes referred to as a biosimilar. In such a situation, third party system 165, such as an insurance provider system, may receive an electronic message indicating a treatment regimen selected by the caregiver. Third party system 165 may identify the selected treatment regimen in the electronic message and apply one or more rule sets for determining whether to approve, deny, or offer an alternative to the selected treatment regimen. The one or more rule sets may define policies for the insurance provider and identifications of treatments that are to be approved. The rule sets may also correlate alternative versions of treatments that would be approved, such as biosimilar treatments or medications. In some embodiments, the rule sets may include tables or logic for identifying alternative dosages for the requested treatment regimen. If the third party system 165 identifies a biosimilar or other modification to the treatment regimen, the system may return a message to processor 140 indicating the request to accept the biosimilar or change, causing processor 140 to generate and display a biosimilar GUI 1400, as illustrated in FIG. 14. GUI 1400 may include an identification of the biosimilar or other change requested by the insurance provider system, and optionally include any messages provided in the response message received from the insurance provider system. Displayed messages may include information the insurance provider system has appended to the response message, based on the rule sets or logic applied to the treatment regimen request. Thereafter, processor may proceed to generate a flowsheet generation GUI 1500, as shown in FIG. 15 and similar to that of FIG. 10.

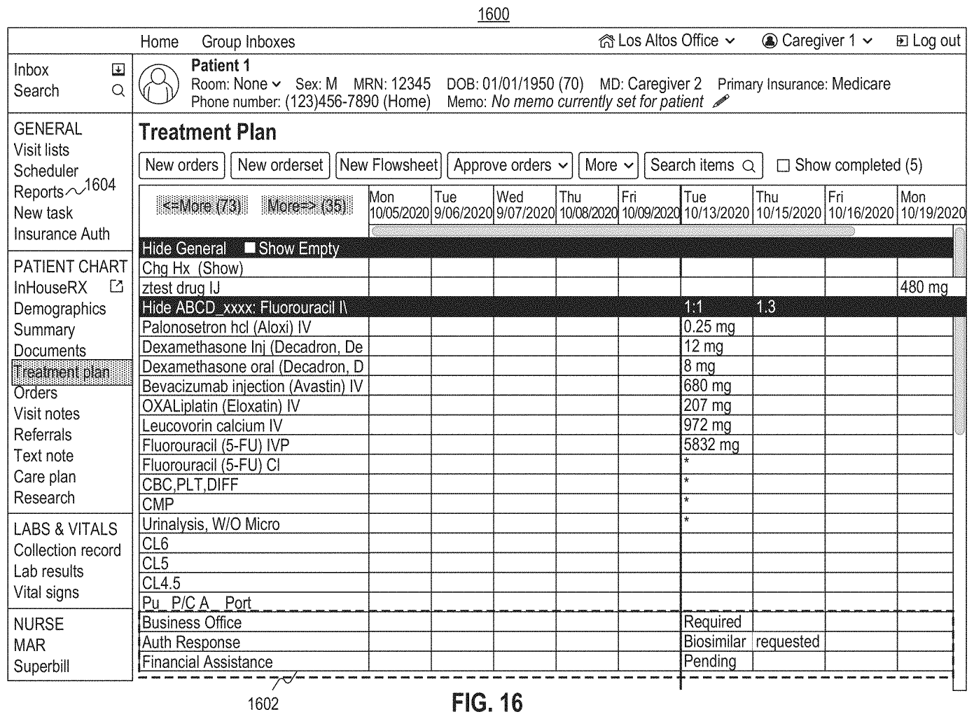

In some embodiments, processor 140 may generate and provide for display a modified version of a treatment plan GUI 1600, as illustrated in FIG. 16. In a status portion 1602 of GUI 1600, processor 1400 may provide for display a notation that a biosimilar has been requested by the insurance provider system in response to the selected treatment regimen is an illustration of another exemplary treatment plan GUI consistent with the present disclosure. In some embodiments, the system may be configured to generate and output one or more reports on insurance provider responses to prior authorization request: e.g., what regimens were approved, denied, and/or substituted. Such reports may be generated in response to selection of a reports tab 1604 as shown in FIG. 16. Similarly, reports may be generated in response to selection of a reports tab 1102 shown in FIG. 11.

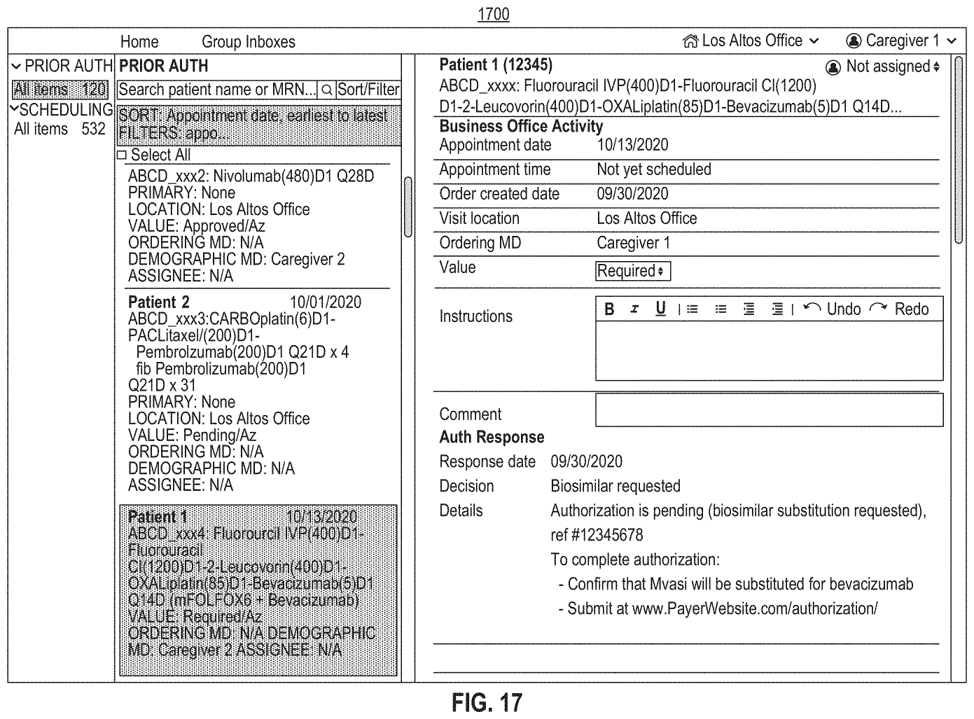

Additionally, processor 140 may generate and provide for display an updated “Auth Response” portion in a prior authorization graphical user interface, such as prior authorization GUI 1700 shown in FIG. 17. As shown, GUI 1700 may include an indication that the insurance provider system has requested a biosimilar or other change to the treatment regimen, and may automatically provide details about the requested change and the necessary steps (if any) to confirm the change and complete the pre-approval process.

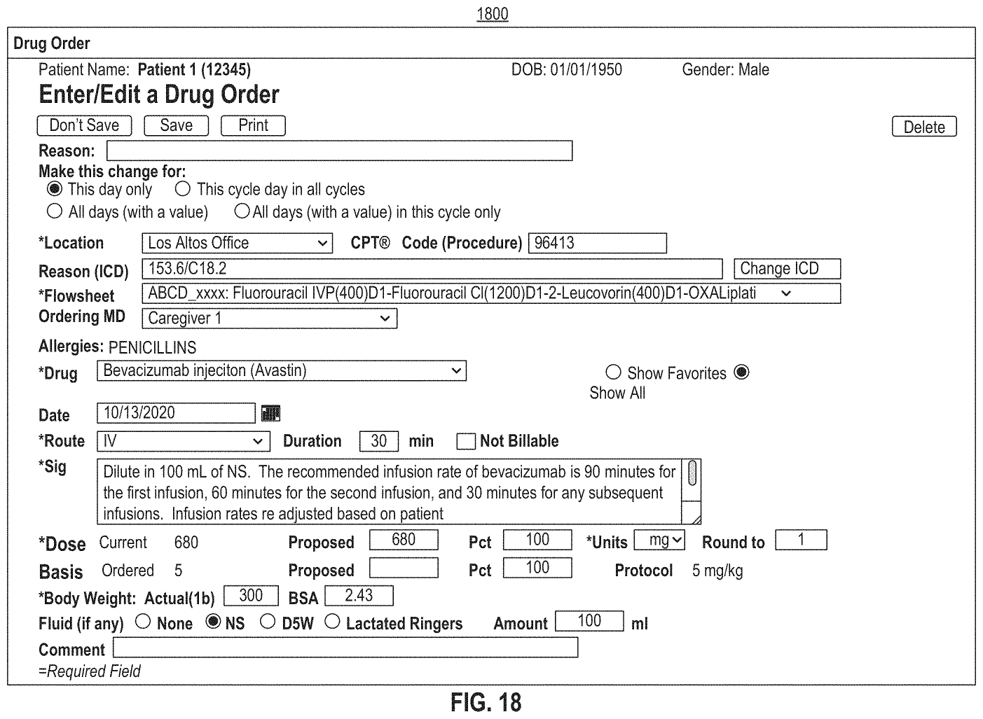

In some embodiments, processor 140 may generate and provide for display a graphical user interface for entering a drug order or editing a drug order, such as drug order GUI 1800 shown in FIG. 18. As shown, GUI 1800 may include information for a drug order that is automatically populated based on the approved treatment regimen for a patient. Processor 140 may provide one or more interactive elements in GUI 1800 for allowing a caregiver to change certain parameters of the drug order, using editable fields, expandable menus, radio buttons, or any other suitable graphical element that allows processor 140 to receive inputs for changes in treatment.

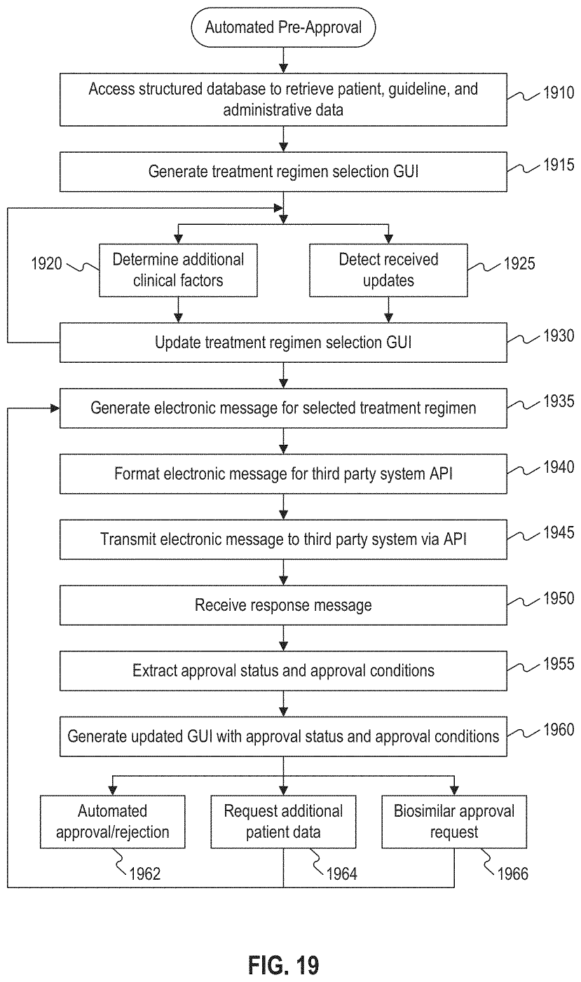

FIG. 19 is a flowchart illustrating an exemplary process for providing automated prior authorization consistent with the present disclosure. In the example described herein, the steps of this process may be performed by processor 140 of system 130. In some embodiments, some steps of the process may be distributed among multiple processing devices, or performed by other processing devices such as client device 160 or processors associated with data sources 110. Therefore, the example described below is not intended to be limiting as to the particular processor performing a given step.

The process may begin at step 1910, in which processor 140 accesses one or more structured or unstructured databases, such as data sources 110, to access patient data 170, guideline data 180, and/or administrative data 190 associated with a patient.

In step 1915, processor 140 may generate and provide for display a graphical user interface for a caregiver to select a treatment regimen, such as the interface depicted in FIG. 7. In some embodiments, processor 140 may identify one or more suggested treatment regimens based on portions of patient data 170 analyzed against guideline data. For example, processor 140 may extract a patient condition and one or more clinical factors in a patient's data, and apply one or more rule sets, decision trees, or other logic from guideline data 180 to the patient data to identify corresponding treatment regimens in the guideline data 180. Processor 140 may then provide a graphical user interface with display of the identified treatment regimens, along with one or more graphical interface elements allowing for selection of a treatment regimen.

In step 1920, processor 140 may determine, based on the patient data 170 and guideline data 180, whether there are additional potential clinical factors for the patient's condition that may be associated with the patient data. If so, processor 140 may provide for display one or more selectable graphical interface elements for adding the additional clinical factors to patient data 170. In step 1925, processor 140 may determine whether a clinician or other user has entered additional clinical factors via the graphical user interface, such as by typing additional clinical factors in an input field of the interface. Processor 140 may also detect selection of the clinical factors determined and displayed in step 1920. In some embodiments, steps 1920 and 1925 may both occur. In other embodiments, steps 1920 or step 1925 may occur.

In step 1930, processor 140 may update the graphical user interface to incorporate additional clinical factors and any associated information identified in steps 1920 and 1925. The updated graphical user interface may be provided for display, and the process may loop to detect any additional changes or detect any additional selections. If no additional changes are detected, the process may proceed once processor 140 determines that a clinician has identified a treatment regimen.

In step 1935, processor 140 may generate, based on the identified treatment regimen, an electronic message for sending to a third party system 165 such as an insurance provider. The message may include portions of patient data 170 that are pertinent to seeking approval of the selected treatment regimen, portions of guideline data 180 associated with the patient condition and selected treatment regimen, and portions of administrative data 190 pertinent to seeking approval of the treatment regimen. In some embodiments, administrative data 190 may include requirements and guidelines previously set by the third party system 165 identifying the types of information that must be provided in order to automatically approve the treatment regimen. Patient data 170 may include the responsive information, and in some embodiments the electronic message may correlate the pertinent patient data 170 with the pertinent guideline data 180 or administrative data 190.

In step 1940, processor 140 may format the electronic message in a format suitable for passing through an API of third party system 165. Processor 140 may format the message based on settings or parameters stored with respect to the third party system 165, in administrative data 190. In step 145, processor 140 may transmit the formatted electronic message to the third party system 165, such as an insurance provider system, via the API.

In step 1950, processor 140 may receive, via the API, a responsive electronic message generated by third party system 165. The responsive message may be generated automatically at the third party system 165 based on the patient data 170, guideline data 180, and administrative data 190 included in the outgoing electronic message. In some embodiments, the responsive message may be generated based on one or more rule sets that configure the third party system 165 to process the received message and associate information in the received electronic message with a responsive status such as an approval, denial, request for a substitution or biosimilar, or request for additional information.

In step 1955, processor 140 may analyze the received responsive electronic message to extract approval status information and any approval conditions provided by the third party system 165. In some embodiments, processor 140 may update one or more electronic records associated with the patient with the extracted approval status information or the approval conditions. Processor 140 may then generate one or more updated graphical user interfaces to reflect the received approval information and any approval conditions, in step 1960. In some embodiments, certain graphical user interfaces may only be displayed depending on certain approval statuses, such as the GUIs shown in FIG. 9 or 14 for automated approval or biosimilar request, respectively.

Following display of the updated graphical user interfaces, if the treatment regimen has been approved with no conditions, then the process may end in with automated approval/rejection 1962. If the approval information includes conditions or requests for additional information (step 1964), then processor 140 may generate and provide for display the appropriate graphical user interfaces at steps 1964 or 1966.

In some embodiments, after additional patient data is requested (step 1964) processor 140 may detect input or receipt of the additional information (not shown in figure). In some embodiments, third party processor 165 may provide indication of a prior authorization pending receipt of the additional information, and processor 140 may automatically indicate approval via the graphical user interface and via one or more messages to third party system 165 after receiving the additional information (not shown in figure). In some embodiments, after receiving input of the additional information from a caregiver, the process may loop back to step 1935 so that processor 140 may generate an updated electronic message to seek final approval or confirm acceptance of the biosimilar. Following final approvals, processor 140 may update a patient EHR in an EHR database, reflecting the selected and approved treatment regimen and any associated conditions or requested biosimilars. In some embodiments, processor 140 may generate the updated electronic message automatically, or in response to selection of one or more interactive user interface elements.

The foregoing description has been presented for purposes of illustration. It is not exhaustive and is not limited to the precise forms or embodiments disclosed. Modifications and adaptations will be apparent to those skilled in the art from consideration of the specification and practice of the disclosed embodiments. Additionally, although aspects of the disclosed embodiments are described as being stored in memory, one skilled in the art will appreciate that these aspects can also be stored on other types of computer readable media, such as secondary storage devices, for example, hard disks or CD ROM, or other forms of RAM or ROM, USB media, DVD, Blu-ray, 4K Ultra HD Blu-ray, or other optical drive media.

Computer programs based on the written description and disclosed methods are within the skill of an experienced developer. The various programs or program modules can be created using any of the techniques known to one skilled in the art or can be designed in connection with existing software. For example, program sections or program modules can be designed in or by means of .Net Framework, .Net Compact Framework (and related languages, such as Visual Basic, C, etc.), Java, Python, R, C++, Objective-C, HTML, HTML/AJAX combinations, XML, or HTML with included Java applets.

Moreover, while illustrative embodiments have been described herein, the scope of any and all embodiments having equivalent elements, modifications, omissions, combinations (e.g., of aspects across various embodiments), adaptations and/or alterations as would be appreciated by those skilled in the art based on the present disclosure. The limitations in the claims are to be interpreted broadly based on the language employed in the claims and not limited to examples described in the present specification or during the prosecution of the application. The examples are to be construed as non-exclusive. Furthermore, the steps of the disclosed methods may be modified in any manner, including by reordering steps and/or inserting or deleting steps. It is intended, therefore, that the specification and examples be considered as illustrative only, with a true scope and spirit being indicated by the following claims and their full scope of equivalents.

1. A system for computer-based automated pre-approvals, the system comprising:
a memory device storing instructions;
a network interface; and
at least one processing device in communication with the memory device and the network interface, the at least one processing device configured to execute the stored instructions to:
access, via the network interface and from at least one database, patient data associated with a patient and guideline data associated with a plurality of treatment regimens;
generate a first graphical user interface (GUI) for receiving selection of a treatment regimen for the patient;
automatically generate and provide a first electronic message to a third party system, by:
identifying a subset of the accessed patient data based on the selected treatment regimen and the accessed guideline data associated with the selected treatment regimen, wherein the identified subset of the accessed patient data includes one or more clinical attributes of the patient;
generating, based on the selected treatment regimen and the identified subset of the accessed patient data, the first electronic message for seeking pre-approval of the selected treatment regimen;
formatting the first electronic message for transmission through an application programming interface (API) of the third party system, wherein t formatting is based on one or more parameters for the API stored in administrative data accessed via the network interface, the administrative data comprising parameters for a plurality of different APIs associated with a plurality of third party systems; and
transmitting, via the network interface and the API, the first electronic message to the third party system;
receive, from the third party system via the network interface, a second electronic message;
extract, from the second electronic message, approval information; and
generate a second GUI for providing the extracted approval information.

2. The system of claim 1, wherein the at least one processing device is further configured to execute the stored instructions to access administrative data associated with one or more requirements of the third party system, and generate the first electronic message based on the accessed administrative data.

3. The system of claim 1, wherein
the at least one processing device is further configured to determine a plurality of candidate treatment regimens,
the first GUI presents the plurality of candidate treatment regimens, and the selected treatment regimen is one of the plurality of candidate treatment regimens.

4-5. (canceled)

6. The system of claim 1, wherein responsive to a determination that the approval information includes at least one approval conditions, the at least one processing device is configured to generate a third GUI for requesting additional information.

7. The system of claim 1, wherein responsive to a determination that the approval information includes a request for a biosimilar, the at least one processing device is configured to generate a fourth GUI for requesting acceptance of the biosimilar.

8. The system of claim 1, wherein the at least one processing device is further configured to update an electronic health record based on the selected treatment regimen and the approval information.

9. A computer-implemented method for automated pre-approval, the method comprising:
accessing, via a network interface and from at least one database, patient data associated with a patient and guideline data associated with a plurality of treatment regimens;
generating a first graphical user interface (GUI) for receiving selection of a treatment regimen for the patient;
automatically generating and providing a first electronic message to a third party system, by:
identifying a subset of the accessed patient data based on the selected treatment regimen and the accessed guideline data associated with the selected treatment regimen, wherein the identified subset of the accessed patient data includes one or more clinical attributes of the patient;
generating, based on the selected treatment regimen and the identified subset of the accessed patient data, the first electronic message for seeking pre-approval of the selected treatment regimen;
formatting the first electronic message for transmission through an application programming interface (API) of the third party system, wherein the formatting is based on one or more parameters for the API stored in administrative data accessed via the network interface, the administrative data comprising parameters for a plurality of different APIs associated with a plurality of third party systems; and
transmitting, via the API, the first electronic message to the third party system;
receiving, from the third party system, a second electronic message;
extracting, from the second electronic message, approval information; and
generating a second GUI for providing the extracted approval information.

10. The method of claim 9, wherein the at least one processing device is further configured to execute the stored instructions to access administrative data associated with one or more requirements of the third party system, and generate the first electronic message based on the accessed administrative data.

11. The method of claim 9, further comprising: determining a plurality of candidate treatment regimens,
wherein the first GUI presents the plurality of candidate treatment regimens, and the selected treatment regimen is one of the plurality of candidate treatment regimens.

12-13. (canceled)

14. The method of claim 9, further comprising:
responsive to a determination that the approval information includes at least one approval conditions, generating a third GUI for requesting additional information.

15. The method of claim 9, further comprising:
responsive to a determination that the approval information includes a request for a biosimilar, generating a fourth GUI for requesting acceptance of the biosimilar.

16. The system of claim 9, further comprising updating an electronic health record based on the selected treatment regimen and the approval information.

17. A non-transitory computer readable medium storing instructions that, when executed, cause at least one processing device to perform operations for computer-based automated pre-approval, the operations comprising:
accessing, via a network interface and from at least one database, patient data associated with a patient and guideline data associated with a plurality of treatment regimens;
generating a first graphical user interface (GUI) for receiving selection of a treatment regimen for the patient;
automatically generating and providing a first electronic message to a third party system, by:
identifying a subset of the accessed patient data based on the selected treatment regimen and the accessed guideline data associated with the selected treatment regimen, wherein the identified subset of the accessed patient data includes one or more clinical attributes of the patient;
generating, based on the selected treatment regimen and the identified subset of the accessed patient data, the first electronic message for seeking pre-approval of the selected treatment regimen;
formatting the first electronic message for transmission through an application programming interface (API) of the third party system, wherein the formatting is based on one or more parameters for the API stored in administrative data accessed via the network interface, the administrative data comprising parameters for a plurality of different APIs associated with a plurality of third party systems; and
transmitting, via the API, the first electronic message to the third party system;
receiving, from the third party system, a second electronic message;
extracting, from the second electronic message, approval information; and
generating a second GUI for providing the extracted approval information.

18. The system of claim 1, wherein the at least one processing device is further configured to dynamically update, using a machine learning algorithm, the accessed guideline data associated with the selected treatment regimen based on the received second electronic message.

19. The method of claim 9, further comprising dynamically updating, using a machine learning algorithm, the accessed guideline data associated with the selected treatment regimen based on the received second electronic message.

20. The non-transitory computer readable medium of claim 17, the operations further comprising dynamically updating, using a machine learning algorithm, the accessed guideline data associated with the selected treatment regimen based on the received second electronic message.
